(19) United States
(12) Patent Application Publication
(10) Pub. No.: US 2009/0305970 A1
(43) Pub. Date: Dec. 10, 2009

(54) Toxin-Like Polypeptides, Polynucleotides Encoding Same and Uses Thereof

(76) Inventors: Michal Linial, Jerusalem (IL); Alex Inberg, Gedera (IL); Noam Kaplan, Jerusalem (IL)

Correspondence Address: MARTIN D. MOYNIHAN d/b/a PRTSI, INC., P.O. BOX 16446, ARLINGTON, VA, 22215, US

(21) Appl. No.: 12/086,116

(22) PCT Filed: Dec. 12, 2006
(86) PCT No.: PCT/IL2006/001433

§ 371 Date: Apr. 23, 2009

Related U.S. Application Data
(60) Provisional application No. 60/749,099, filed on Dec. 12, 2005.
(60) Provisional application No. 60/777,515, filed on Mar. 1, 2006.

(51) Int. Cl. A61K 38/16; C12N 15/11; C07K 14/435; A01N 33/00

(52) U.S. Cl. 514/12; 536/23.5; 530/324; 530/326; 530/350

(57) ABSTRACT

An isolated polynucleotide is disclosed comprising a nucleic acid sequence encoding a polypeptide which comprises an amino acid sequence at least 90% identical to a sequence as set forth in SEQ ID NO: 1, wherein the polypeptide comprises an ion channel modulatory activity. Polypeptides and uses thereof are also disclosed.

### FIELD AND BACKGROUND OF THE INVENTION

The present invention relates to novel toxin-like polypeptides and polynucleotides encoding same.

The animal kingdom includes more than 100,000 venomous species spread through major phyla. The ‘venom’ is the sum of all natural venomous substances produced in the animal kingdom.

Each individual venom is a unique cocktail of often more than 100 different peptides and proteins, making the venom a source of millions of peptides and proteins naturally tailored to act on innumerable targets in the ‘recipient’ including ion channels, receptors and enzymes within cells and on the plasma membrane. Thus, toxin peptides have been classified according to numerous functions including ion channel inhibitors (ICIs), phospholipases, protease inhibitors, disintegrins and defensins.

It has been demonstrated that venom peptides and proteins constitute a unique source of drugs and drug leads for the treatment of broad range of diseases. For example, peptide toxins that function as channel blockers are ideal drugs for pain therapy. Ziconotide, a synthetic form of MVIIA ω-conotoxin is a voltage-gated Ca²⁺ ion channel inhibitor from Conus magus, is delivered directly to the patient's central nerve system for treatment of chronic pain.

Toxin peptides as drugs have also been designed to address diseases such as cancer, autoimmune diseases, allergies, hypertension, infectious diseases and neurodegenerative disorders—see e.g. Lewis R J, Garcia M L, Nat Rev Drug Discov. 2003.

As well as being varied in their biochemical function, toxins are extremely varied in their sequences and structure as well. For example, even specific groups of ICIs, which inhibit the same target channels, often vary in sequence and structural fold [Mouhat S, et al., Biochem J 2004, 378(Pt 3):717-726].

Therefore, the high-level functionality of these proteins as toxins is computationally unclassifiable by state of the art sequence-based methods e.g. local sequence alignment search tools such as BLAST or FASTA. In addition, due to their short size, toxin peptides are often unidentified during large scale genome annotation projects.

Many of the functions and structures of animal peptide toxins (APTs) are not exclusive to APTs. Instances of APT and APT-like proteins that act in non-venom contexts have been reported. One of the most striking examples is that of Lynx1 and SLURP-1 [Chimienti F et al., Mol Genet. 2003, 12(22):3017-3024; Ibanez-Tallon I, et al., Neuron 2002, 33(6):893-903; Miwa J. M. Neuron 1999, 23(1):105-114]. These are human proteins that not only possess similarity to snake α-neurotoxins, but also modulate nicotinic acetylcholine receptors (nAChRs) as do α-neurotoxins. Mutation in the gene of SLURP-1 causes Mal de Meleda disease, a skin disease that results from an improper activation of TNF-α. Lynx1 has recently been shown to affect neuronal activity and survival in the CNS. These reported instances suggest that, in evolutionary terms, many toxins are homologs of endogenous non-venom proteins and may have been recruited to act in a venom context [Fry B. G., Genome Res 2005, 15(3):403-420] or vice versa. Considering these findings, it is conceivable that there exist additional unknown APT-like proteins, which adopt structural and functional principles that are similar to those of APTs.

In light of the sequential, structural and functional diversity of APTs, there is a need for, and it would be highly advantageous to have novel methods of identifying animal peptide toxins which do not rely solely on sequence and sporadic discovery from venomous glands data.

### SUMMARY OF THE INVENTION

According to one aspect of the present invention there is provided an isolated polynucleotide comprising a nucleic acid sequence encoding a polypeptide which comprises an amino acid sequence as set forth in SEQ ID NO: 1.

According to another aspect of the present invention there is provided an isolated polynucleotide comprising a nucleic acid sequence encoding a polypeptide which comprises an amino acid sequence at least 90% identical to a sequence as set forth in SEQ ID NO: 1, wherein the polypeptide comprises an ion channel modulatory activity.

According to yet another aspect of the present invention there is provided an isolated polynucleotide comprising a nucleic acid sequence encoding a polypeptide which comprises an amino acid sequence at least 90% identical to a sequence selected from the group consisting of SEQ ID NOs: 3-12, wherein the polypeptide comprises an ion channel modulatory activity.

According to still another aspect of the present invention there is provided an isolated polypeptide comprising an amino acid sequence as set forth in SEQ ID NO: 1.

According to an additional aspect of the present invention there is provided an isolated polypeptide comprising an amino acid sequence selected from the group consisting of SEQ ID NOs: 3-12.

According to yet an additional aspect of the present invention there is provided an isolated polypeptide comprising an amino acid sequence at least 90% identical to a sequence as set forth in SEQ ID NO: 1, wherein the polypeptide comprises an ion channel modulatory activity.

According to still an additional aspect of the present invention there is provided a molecule comprising the isolated polypeptides of the present invention, the polypeptides being attached to an affinity moiety.

According to a further aspect of the present invention there is provided a nucleic acid construct comprising any of the polynucleotides of the present invention.

According to yet a further aspect of the present invention there is provided a cell comprising the nucleic acid construct of the present invention

According to still a further aspect of the present invention, there is provided a pharmaceutical composition comprising a pharmaceutically acceptable carrier and as an active ingredient an isolated polypeptide, which comprises an amino acid sequence having a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue.

According to still a further aspect of the present invention, there is provided a pesticidal composition comprising an agriculturally acceptable carrier and as an active ingredient an isolated polypeptide, wherein an amino acid sequence of the isolated polypeptide confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue.

According to still a further aspect of the present invention, there is provided a use of an isolated polypeptide, wherein an amino acid sequence of the isolated polypeptide confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue for the manufacture of a medicament identified for the treatment of a nerve disease or disorder.

According to still a further aspect of the present invention, there is provided a use of an isolated polypeptide, wherein an amino acid sequence of the isolated polypeptide confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue for the manufacture of a medicament identified for a cosmetic treatment.

According to still a further aspect of the present invention, there is provided a method of controlling or exterminating an insect, the method comprising applying to the insect an insecticidally effective amount of an isolated polypeptide, wherein an amino acid sequence of the isolated polypeptide confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue, thereby controlling or exterminating the insect.

According to still a further aspect of the present invention, there is provided a method of treating a nerve disease or disorder, the method comprising administering to a subject in need thereof a therapeutically effective amount of a polypeptide comprising an amino acid sequence, wherein the amino acid sequence confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue, thereby treating the nerve disease or disorder.

According to further features in preferred embodiments of the invention described below, the amino acid sequence is as set forth in SEQ ID NO: 2

According to still further features in the described preferred embodiments, the nucleic acid comprises a sequence as set forth in SEQ ID NO: 13 or SEQ ID NO: 14.

According to still further features in the described preferred embodiments, the nucleic acid sequence is selected from the group consisting of SEQ ID NOs: 15-17.

According to still further features in the described preferred embodiments, the polypeptide comprises an amino acid sequence as set forth in SEQ ID NO: 2.

According to still further features in the described preferred embodiments, the affinity moiety is selected from the group consisting of an antibody, a receptor ligand and a carbohydrate.

According to still further features in the described preferred embodiments, the nucleic acid construct further comprises a cis regulatory element for regulating expression of the polynucleotides of the present invention.

According to still further features in the described preferred embodiments, the nerve disease or disorder, is a CNS disease or disorder.

According to still further features in the described preferred embodiments, the nerve disease or disorder is a peripheral nerve disease or disorder.

According to still further features in the described preferred embodiments, the CNS disease or disorder is selected from the group consisting of a pain disorder, a motion disorder, a dissociative disorder, a mood disorder, an affective disorder, a neurodegenerative disease or disorder, an addictive disorder and a convulsive disorder.

According to still further features in the described preferred embodiments, the CNS disease or disorder is selected from the group consisting of Parkinson's, Multiple Sclerosis, Huntington's disease, action tremors and tardive dyskinesia, panic, anxiety, depression, Alzheimer's and epilepsy.

According to still further features in the described preferred embodiments, the peripheral nerve disease or disorder is selected from the group consisting of a hereditary neuropathy, a mononeuritis multiplex, a mononeuropathy, a muscle stimulation disorder, a neuromuscular junction disorder, a plexus disorder, a polyneuropathy, a spinal muscular atrophy and a thoracic outlet syndrome.

According to still further features in the described preferred embodiments, the X₂ is a hydrophobic amino acid, X₅ is a small amino acid, X₆ is a turnlike amino acid, X₉ is a hydrophobic amino acid, X₁₁ is a polar amino acid, X₁₂ is a turnlike amino acid, X₁₄ is a polar amino acid, X₁₇ is a small amino acid, X₂₀ is a turnlike amino acid, X₂₃ is a hydrophobic amino acid, X₂₅ is an aromatic amino acid, X₂₈ is a positive amino acid and X₃₀ is a hydrophobic amino acid.

According to still further features in the described preferred embodiments, the X₂ is a hydrophobic amino acid, X₅ is glycine, X₆ is a polar amino acid, X₉ is a hydrophobic amino acid, X₁₀ is a turnlike amino acid, X₁₁ is a polar amino acid, X₁₂ is a turnlike amino acid, X₁₄ is a polar amino acid, X₁₇ is a small amino acid, X₂₀ is a turnlike amino acid, X₂₁ is a turnlike amino acid, X₂₃ is a hydrophobic amino acid, X₂₄ is a small amino acid, X₂₅ is an aromatic amino acid, X₂₆ is a turnlike amino acid, X₂₈ is a positive amino acid and X₃₀ is an aliphatic amino acid.

According to still further features in the described preferred embodiments, the X₂ is a small amino acid, X₃ is a turn-like amino acid, X₄ is a small amino acid, X₅ is glycine, X₆ is a polar amino acid, X₇ is a hydrophobic amino acid, X₉ is a hydrophobic amino acid, X₁₀ is a small amino acid, X₁₁ is a polar amino acid, X₁₂ is a small amino acid, X₁₄ is a polar amino acid, X₁₇ is serine, X₂₀ is a small amino acid, X₂₁ is a small amino acid, X₂₃ is a hydrophobic amino acid, X₂₄ is a small amino acid, X₂₅ is an aromatic amino acid, X₂₆ is a tiny amino acid, X₂₇ is a hydrophobic amino acid, X₂₈ is a positive amino acid and X₃₀ is valine.

According to still further features in the described preferred embodiments, the X₂ is a tiny amino acid, X₃ is a turn-like amino acid, X₄ is a small amino acid, X₅ is glycine, X₆ is a negative amino acid, X₇ is an aromatic amino acid, X₉ is an aliphatic amino acid, X₁₀ is a small amino acid, X₁₁ is a charged amino acid, X₁₂ is a small amino acid, X₁₄ is a negative amino acid, X₁₇ is serine, X₂₀ is a small amino acid, X₂₁ is a small amino acid, X₂₃ is leucine, X₂₄ is an alcoholic amino acid, X₂₅ is tyrosine, X₂₆ is a tiny amino acid, X₂₇ is a hydrophobic amino acid, X₂₈ is lysine and X₃₀ is valine.

According to still further features in the described preferred embodiments, the X₂ is a tiny amino acid, X₃ is a turn-like amino acid, X₄ is a small amino acid, X₅ is glycine, X₆ is Glutamic acid, X₇ is an aromatic amino acid, X₉ is lysine, X₁₀ is an alcoholic amino acid, X₁₁ is histidine, X₁₂ is a small amino acid, X₁₄ is a negative amino acid, X₁₇ is serine, X₂₀ is a small amino acid, X₂₁ is a tiny amino acid, X₂₃ is leucine, X₂₄ is an alcoholic amino acid, X₂₅ is tyrosine, X₂₆ is a tiny amino acid, X₂₇ is a turn-like amino acid, X₂₈ is lysine and X₃₀ is valine.

According to still further features in the described preferred embodiments, the X₅ is glycine, X₆ is a turnlike amino acid, X₉ is a hydrophobic amino acid, X₁₀ is a turnlike amino acid, X₁₁ is a polar amino acid, X₁₂ is a turnlike amino acid, X₁₇ is a polar amino acid, X₂₅ is an aromatic amino acid, X₂₆ is an turnlike amino acid and X₂₈ is a polar amino acid.

According to still further features in the described preferred embodiments, the X₂ is a hydrophobic amino acid, X₅ is glycine, X₆ is a turnlike amino acid, X₉ is a hydrophobic amino acid, X₁₀ is a turnlike amino acid, X₁₁ is a polar amino acid, X₁₂ is a small amino acid, X₁₄ is a polar amino acid, X₁₇ is a small amino acid, X₂₀ is a turnlike amino acid, X₂₃ is a hydrophobic amino acid, X₂₅ is tyrosine, X₂₆ is a small amino acid, X₂₈ is a positive amino acid and X₃₀ is a hydrophobic amino acid.

According to still further features in the described preferred embodiments, the X₂ is a hydrophobic amino acid, X₃ is a small amino acid, X₄ is a hydrophobic amino acid, X₅ is glycine, X₆ is a polar amino acid, X₉ is a hydrophobic amino acid, X₁₀ is a turnlike amino acid, X₁₁ is a polar amino acid, X₁₂ is a small amino acid, X₁₄ is a polar amino acid, X₁₇ is a small amino acid, X₂₀ is a turnlike amino acid, X₂, is a turnlike amino acid, X₂₃ is a hydrophobic amino acid, X₂₄ is a small amino acid, X₂₅ is tyrosine, X₂₆ is a tiny amino acid, X₂₈ is a positive amino acid and X₃₀ is a small amino acid.

According to still further features in the described preferred embodiments, the X₂ is a turnlike amino acid, X₃ is a small amino acid, X₄ is a hydrophobic amino acid, X₅ is glycine, X₆ is a polar amino acid, X₉ is a hydrophobic amino acid, X₁₀ is a small amino acid, X₁₁ is a polar amino acid, X₁₂ is a small amino acid, X₁₄ is a polar amino acid, X₁₇ is a small amino acid, X₂₀ is a turnlike amino acid, X₂₁ is a polar amino acid, X₂₃ is a hydrophobic amino acid, X₂₄ is a small amino acid, X₂₅ is tyrosine, X₂₆ is a tiny amino acid, X₂₇ is a small amino acid, X₂₈ is a positive amino acid and X₃₀ is an aliphatic amino acid.

According to still further features in the described preferred embodiments, the X₂ is a tiny amino acid, X₃ is a tiny amino acid, X₄ is a small amino acid, X₅ is glycine, X₆ is a negative amino acid, X₇ is a polar amino acid, X₉ is an aliphatic amino acid, X₁₀ is a small amino acid, X₁₁ is a small amino acid, X₁₂ is a small amino acid, X₁₄ is a negative amino acid, X₁₇ is serine, X₂₀ is a small amino acid, X₂₁ is a small amino acid, X₂₃ is a hydrophobic amino acid, X₂₄ is an alcoholic amino acid, X₂₅ is tyrosine, X₂₆ is alanine, X₂₇ is a small amino acid, X₂₈ is lysine and X₃₀ is valine.

According to still further features in the described preferred embodiments, the isolated polypeptide comprises any of the sequences selected from the group consisting of SEQ ID NOs: 1-12 and SEQ ID NOs: 20-30.

According to yet another aspect of the present invention there is provided an isolated polynucleotide comprising a nucleic acid sequence encoding a polypeptide which comprises an amino acid sequence as set forth in SEQ ID NOs: 31-35.

According to yet another aspect of the present invention there is provided an isolated polynucleotide comprising a nucleic acid sequence encoding a polypeptide which comprises an amino acid sequence at least 90% identical to an amino acid sequence as set forth in SEQ ID NOs: 31-35, wherein the polypeptide comprises an ion channel modulatory activity.

According to still further features in the described preferred embodiments, the nucleic acid is selected from the group consisting of SEQ ID NOs: 36-38.

According to yet another aspect of the present invention there is provided an isolated polypeptide comprising an amino acid sequence selected from a group consisting of SEQ ID NOs: 31-35.

According to yet another aspect of the present invention there is provided an isolated polypeptide comprising an amino acid sequence at least 90% identical to an amino acid sequence as set forth in SEQ ID NOs: 31-35, wherein the polypeptide comprises an ion channel modulatory activity.

According to yet another aspect of the present invention there is provided an isolated polynucleotide comprising a nucleic acid sequence encoding a polypeptide which comprises an amino acid sequence as set forth in SEQ ID NOs: 39-46 and 57-59.

According to yet another aspect of the present invention there is provided an isolated polynucleotide comprising a nucleic acid sequence encoding a polypeptide which comprises an amino acid sequence at least 90% identical to an amino acid sequence as set forth in SEQ ID NOs: 39-46 and 57-59, wherein the polypeptide comprises an ion channel modulatory activity.

According to still further features in the described preferred embodiments, the nucleic acid is selected from the group consisting of SEQ ID NOs: 47-56 and 60-62.

According to yet another aspect of the present invention there is provided an isolated polypeptide comprising an amino acid sequence selected from a group consisting of SEQ ID NOs: 39-46 and 57-59.

According to yet another aspect of the present invention there is provided an isolated polypeptide comprising an amino acid sequence at least 90% identical to an amino acid sequence as set forth in SEQ ID NOs: 39-46 and 57-59, wherein the polypeptide comprises an ion channel modulatory activity.

The present invention successfully addresses the shortcomings of the presently known configurations by providing novel toxin-like polypeptides and polynucleotides encoding same.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a score distribution of predictions. The score distribution was predicted on a non-redundant set of all 29554 SwissProt proteins shorter than or equal to 150 aa.

FIG. 2 is a score distribution of selected biological groups. The horizontal axis represents the mean prediction score. Thick red vertical lines represent median values of each group. The groups ‘ICI’, ‘Toxin’, ‘Neurotoxin’ and ‘Antibacterial’ are based on UniProt keywords. All groups except the top one (ICI) include only proteins that were not part of the training set. The groups ‘ICI’, ‘Snake toxin’, ‘Neurotoxin’ and ‘Beta defensin’ receive mostly positive scores. The ‘Toxin’ and ‘Venom protein’ groups tend to be positive but the separation is weaker. The ‘Antibacterial’ group is mostly negative, but there is clearly a significant portion of positive instances (note that ‘Beta defensin’ is a subset of group). The ‘E6’ (E6 early regulatory protein), ‘L36’ (ribosomal protein L36) and ‘Gonadotropin’ groups are known to be cysteine-rich but are clearly predicted negative.

FIG. 3 is the nucleotide and amino acid sequence of OCLP1. Yellow and green backgrounds represent the first and second exons. Blue amino acids represent the putative location of the signal peptide (predicted by SignalP). Red amino acids represent the mature peptide and black letters represent an extended unstructured tail. Note the exon positioning in which the first exon ends just before the second cysteine of the putative mature peptide.

FIG. 4 is a multiple sequence alignment of OCL proteins. A-E indicates repeats within the OCLP1 protein homologs. Highly conserved positions are highlighted. Cysteines appear in bold. Disulfide connectivity is shown beneath the alignment. OCLP1 homologs are noted in species names only. A-E indicates OCL repeats. Only the OCL region is shown. Note the YANRC sequence which is shared only by OCLP1, Ado1, Ptu1 and Iob1.

FIG. 5 is a model of OCLP1. Side chains are shown for the 6 conserved cysteines (disulfide bonds appear in yellow) and for the conserved positions 25-28 that are unique to OCLP1 and the assassin bug toxins. Model was created using SDPMOD (homology modeled after 1LMR).

FIG. 6 is a photograph illustrating the expression of OCLP1. Products of RT-PCR using total RNA extracted from bee brain and head following separation on 1.5% agarose gel are shown. The short version (169 nt) is the OCLP1 mature form and the long version (240 nt) is the full length transcript. The similar expression level in head and brain indicates that OCLP1 is expressed in the brain rather than tissues outside the brain, such as the salivary gland.

FIG. 7 is an amino acid sequence of the Anopheles gambiae OCLP1 homolog. Blue amino acids represent the putative location of the signal peptide (predicted by SignalP). Red amino acids represent the locations of the OCL repeats. Note that the exons are positioned similarly relatively to the OCL repeats, with each of the exons ending before the second cysteine of an OCL repeat.

FIG. 8 is a multiple sequence alignment of Raalin and putative orthologs. Positions that are identical in at least 5 sequences are highlighted. Note that this alignment shows only the putative mature peptide region. Homologs are noted in species names only.

FIG. 9 is an overview of the prediction procedure. A protein sequence is transformed into a vector of 545 features. The vector is independently sent to 10 boosted stump classifiers, each of which produces a numerical result. The mean of the results is the final (mean) score. The standard deviation of the score indicates how much the 10 sub-classifiers agree with one another.

FIGS. 10A-B are graphs and photographs of analysis of the OCLP1 polypeptide of the present invention following cleavage of the expressed protein from its tag, recovering the free toxin after a refolding protocol, a concentrating step by size exclusion procedure and enzymatic processing. FIG. 10A is a photograph of a Coomassie stained gel of the proteins purified from bacteria following expression of the OCLP1 construct. FIG. 10B is a readout of the Maldi T of analysis confirming the identity of a major band of 3031 dalton, identical to the expected size of the protein.

FIGS. 11A-D are schematic representations and graph recordings depicting the change in current following injection of the OCLP1 polypeptide (SEQ ID NO: 1) into Xenopus laevis. FIG. 11A is a schematic representation of a Ca2+ channel. FIG. 11B depicts the evolutionary relationship of the various Ca2+ channels by the homology tree. FIGS. 11C-D are graphs illustrating the current recorded by whole cell recording in Xenopus laevis Stage V or VI oocytes. α_{1A} calcium channel cDNA of the N type (FIG. 11C) and R type (FIG. 11D) was injected into the nuclei of the oocytes with essential auxiliary subunits. In control experiments, oocytes were either left uninjected (or injected with auxiliary subunits alone, marked in red). Whole-cell currents were measured with two-electrode voltage clamp 3 days after injection. The total concentration of cDNA, (A 260 nm), was constant in each case and the results were normalized by the wild-type amplitude recorded. An average of 8 oocytes were injected with the Bee OCLP1 expressed toxin (SEQ ID NO: 1). Up to 10% change in the current is reported for the injected N-type oocytes (compare black to red lines). The change in tail current is indicative of an effect on calcium channel N-type or on a specialized alternative spliced variant of it. FIG. 11D: 8 individual recordings of oocytes and controls injected with R-type channel—no effect of OCLP1 was recorded above background noise (marked in black line).

FIGS. 12A-D are photographs and photomicrographs illustrating the effect of differentiation on the expression of ANLP-1. Cells were prepared from the cell line P19. FIG. 12A: Cells cultured in monolayer as undifferentiated cells. FIG. 12B: at day 1-4 the cells were exposed to RA and were grown as cell aggregates with RA. FIG. 12C: Cells 48 hrs following replating produced neurites and acquired the properties of neurons of the Central Nerve system. FIG. 12D: Expression of ANLP-1 in cells at the different phases of differentiation (UN, undifferentiated are cells as in FIG. 12A. The RNA used for the RT-PCR was extracted from the P19 neurons at the indicated days of differentiation. Diff, refer to day 6 of differentiation (as in FIG. 12C). A representative result is shown for the expression. Expression levels of ribosomal L19 gene were used for calibration and were identical in all samples (not shown). Each set of primers was tested 3 independent times with <10% variation between independent experiments.

### DESCRIPTION OF THE PREFERRED EMBODIMENTS

The present invention relates to polypeptides, (and polynucleotides encoding same) which comprise structural properties similar to those of known ion channel inhibitors.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Animal peptide toxins (APT) are short proteins that appear in animal venom and are aimed at inflicting harm to the organism on which the venom acts. Sporadic instances of endogenous toxin-like peptides that function in non-venom context have been previously reported. APTs are extremely varied in terms of function and include ion channel inhibitors (ICIs), phospholipases, protease inhibitors, disintegrins, defensins and other biological groups. Even specific groups of ICIs, which inhibit the same target channels, often vary in sequence and structural fold. However, it has been noted that a common characteristic of many such toxins is their apparent structural stability.

In light of the sequential, structural and functional diversity of APTs, it has proven impossible up until presently to find a global characterization of APTs by standard automatic classification methods.

Whilst conceiving the present invention, the present inventors utilized machine learning methodology, based on sequence-derived features and guided by the notion of structural stability, in order to conduct a large-scale search for toxin and toxin-like proteins.

The present inventors trained the machine to identify toxin-like peptides using proteins classified as ion channel inhibitors. When the classifier was applied to a non-redundant set of all 29554 SwissProt proteins shorter than or equal to 150 aa, several different APT-related functional categories were detected (ICIs, phospholipases, disintegrins, protease inhibitors, etc.) indicating that the classifier is apparently able to correctly produce a non-trivial characterization of APT and APT-like proteins. In addition, the results showed that most highly over-represented groups were APT-related—Table 1 of the Examples section hereinbelow.

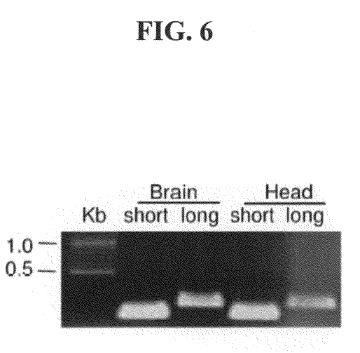

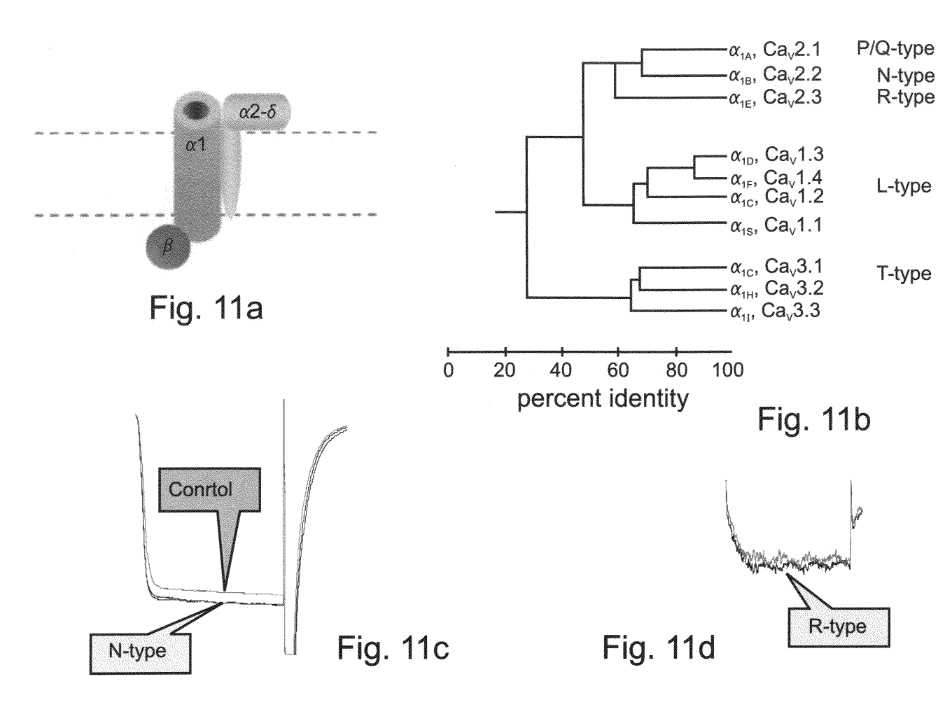

Application of the method of the present invention to insect and mammalian sequences revealed novel toxin-like polypeptide families. Accordingly, two novel bee polypeptides were identified, named by the present inventors as OCLP-1 (co-conotoxin-like) and Raalin. OCLP1 showed a high structural and sequence similarity to ion channel inhibitors that are expressed in cone snail and assassin bug venom. OCLP1 was shown to be expressed in the bee brain and head by RT-PCR (FIG. 6) and following injection into fish, OCLP1 was shown to reversibly cause paralysis thereof. OCLP1 injection into Xenopus oocytes previously transfected with ion channels known to be associated with pain (Ca channel α₁, α₂, and β subunits), caused a consistent change of ˜10% in current flow, indicating that OCLP1 may have an effect on pain (FIGS. 11A-D).

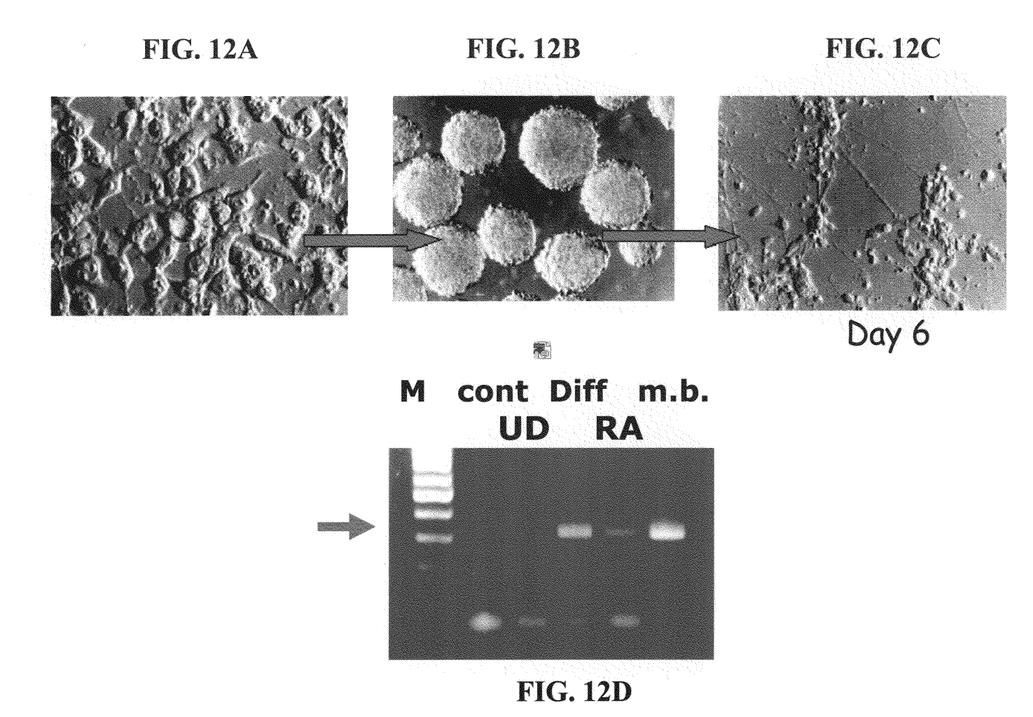

In addition, eight novel mouse polypeptides and three novel human homologues were identified when the classifier was used to screen the 5154 sequences which are comprised in the FANTOM database (http://fantom.gsc.riken.gojp/). One of the mouse polypeptides (ANLP-1) was shown to be upregulated in P19 cells following differentiation into neurons but was unexpressed before the differentiation programe was induced. Upregulation was achieved by retinoic acid —FIGS. 12A-D. mANLP-3 was also induced in neuronal RNA (from mature mouse brain). Without being bound to theory, it is believed that these features testify to the functionality of these novel ANLP-1 polypeptides.

Thus, according to one aspect of the present invention, there is provided an isolated polypeptide comprising an amino acid sequence at least 90% identical to a sequence as set forth in SEQ ID NO: 1, wherein said polypeptide comprises an ion channel modulatory activity.

As used herein, the phrase “ion channel” refers to one or more polypeptides having the ability to transportions across biological membranes. Ion channels are classified upon their ion specificity, biological function, regulation or molecular structure. Examples of ion channels include, but are not limited to voltage-gated ion channels, Gap-junction ion channels, ligand-gated ion channels, heat-activated ion channels, intracellular ion channels, ion channels gated by intracellular ligands such as cyclic nucleotide-gated channels and calcium-activated ion channels.

The phrase ‘ion channel modulating activity” as used herein, refers to an ability to either up-regulate (i.e. agonist activity) or down-regulate (i.e. antagonist activity) the flow of ions through the ion channel.

The term “polypeptide” as used herein encompasses native polypeptides (either degradation products, synthetically synthesized polypeptides or recombinant polypeptides) and peptidomimetics (typically, synthetically synthesized polypeptides), as well as peptoids and semipeptoids which are polypeptide analogs, which may have, for example, modifications rendering the polypeptides more stable while in a body or more capable of penetrating into cells. Examples of polypeptide modifications are described hereinbelow.

According to a preferred embodiment of this aspect of the present invention, the polypeptide comprises an amino acid sequence as set forth in SEQ ID NO: 1. This sequence encodes at least the active part (i.e. comprises biological activity) of the full length protein expressed in the bee brain, also referred to herein as active OCLP1. According to another embodiment of this aspect of the present invention the polypeptide comprises an amino acid sequence as set forth in SEQ ID NO: 2. This sequence encodes the full length protein, referred to herein as full length OCLP1.

Polypeptides of the present invention also include homologs of the active OCLP1 (e.g., polypeptides which are at least 50%, at least 55%, at least 60%, at least 65%, at least 70%, at least 75%, at least 80%, at least 85%, at least 87%, at least 89%, at least 90%, at least 91%, at least 93%, or more say at least 95% to SEQ ID NO: 1 as determined using BlastP software of the National Center of Biotechnology Information (NCBI) using default parameters).

The homolog may also refer to a deletion, insertion, or substitution variant, including an amino acid substitution, thereof and biologically active polypeptide fragments thereof. For example, it has been shown that between the two cysteines at positions 15 and 20, a deletion of a single amino acid is possible without affecting biological activity [Sasaki et al., 2000, FEBS Letters, Volume 466, Issue 1, Pages 125-129].

Also, the last amino acid may be deleted to generate an active peptide of 27 amino acids (SEQ ID NO: 63), the last two amino acid may be deleted to generate an active peptide of 26 amino acids (SEQ ID NO: 64) and the last three amino acids may be deleted to generate an active peptide of 25 amino acids (SEQ ID NO: 65).

The present invention also contemplates other conservative variations of SEQ ID NO: 1.

The phrase “conservative variation” as used herein refers to the replacement of an amino acid residue by another, biologically similar residue. Examples of conservative variations include the substitution of one hydrophobic residue such as isoleucine, valine, leucine, or methionine for another, or the substitution of one solar residue for another, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide. Typically “essential amino acids” are maintained or replaced by conservative substitutions while non-essential amino acids may be maintained, deleted or replaced by conservative or non-conservative replacements. Generally, essential amino acids are determined by various Structure-Activity-Relationship (SAR) techniques (for example amino acids when replaced by Ala cause loss of activity) are replaced by conservative substitution while non-essential amino acids can be deleted or replaced by any type of substitution. The present inventors have shown that the essential amino acids comprised in SEQ ID NO: 1 include the cysteins at positions 1, 8, 14, 15, and 27 and glycines at positions 5 and 17.

Identification of essential vs. non-essential amino acids in the peptide can be achieved by preparing several peptides candidates in which each amino acid is sequentially replaced by the amino acid Ala (Ala-Scan), or sequentially each amino acid is omitted (omission-scan). This allows to identify the amino acids which modulating activity is decreased by said replacement/omission (“essential”) and which are not decreased by said replacement/omission (non-essential) (Morrison et al., Chemical Biology 5:302-307, 2001). Another option for testing the importance of various peptides is by the use of site-directed mutagenesis. Other Structure-Activity-Relationship techniques may also be used. Another method for identifying essential vs. non-essential amino acids in the peptide is by finding consensus sequences between the protein and its orthologs. Conserved amino acids throughout the animal kingdom suggest that the amino acid may bear relevance to function. Consensus sequences are further described hereinbelow.

It will be appreciated that the present inventors have identified putative orthologs of OCLP1 throughout the insect kingdom, which are also considered within the scope of the present invention. Such orthologs are presented in Table 1 hereinbelow.

TABLE 1

| Organism | SEQ ID NO: |
|---|---|
| Aedes_aegypti_A | SEQ ID NO: 3 |
| Aedes_aegypti_B | SEQ ID NO: 4 |
| Anopheles_funestus_B | SEQ ID NO: 5 |
| Aedes_aegypti_C | SEQ ID NO: 6 |
| Musca_domestica_POI | SEQ ID NO: 7 |
| Heliconius_erato | SEQ ID NO: 8 |
| Manduca_sexta | SEQ ID NO: 9 |
| Schmidtea_mediterranea | SEQ ID NO: 10 |
| Aedes_aegypti_D | SEQ ID NO: 11 |
| Anopheles_funestus_A | SEQ ID NO: 12 |
| Anopheles gambiae E | SEQ ID NO: 20 |
| covalitoxin II | SEQ ID NO: 21 |
| Drosophila melanogaster | SEQ ID NO: 22 |
| Drosophila melanogaster | SEQ ID NO: 23 |
| Anopheles gambiae A | SEQ ID NO: 24 |
| Anopheles gambiae B | SEQ ID NO: 25 |
| Anopheles gambiae C | SEQ ID NO: 26 |
| Anopheles gambiae D | SEQ ID NO: 27 |
| P58608 ADO1_AGRDO | SEQ ID NO: 28 |
| P58609 IOB1_ISYOB | SEQ ID NO: 29 |
| P58609 IOB1_ISYOB | SEQ ID NO: 30 |

Using bioinformatic tools, the present inventors have found consensus sequences for active OCLP1 and its orthologs. As mentioned hereinabove, these consensus sequences may also serve as indications for essential and non essential amino acids and thus may be used a tool for selecting a particularly preferred amino acid sequence.

Thus according to one embodiment, the amino acid sequence of the OCLP1 polypeptides of the present invention confers about 90% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X_{30.}

where X₁ is cysteine, X₂ is a hydrophobic amino acid, X₅ is a small amino acid, X₆ is a turnlike amino acid, X₈ is cysteine, X₉ is a hydrophobic amino acid, X₁₁ is a polar amino acid, X₁₂ is a turnlike amino acid, X₁₄ is a polar amino acid, X₁₅ is cysteine, X₁₆ is cysteine, X₁₇ is a small amino acid, X₂₀ is a turnlike amino acid, X₂₂ is cysteine, X₂₃ is a hydrophobic amino acid, X₂₅ is an aromatic amino acid, X₂₈ is a positive amino acid, X₂₉ is cysteine, and X₃₀ is a hydrophobic amino acid.

As used herein, the phrase “hydrophobic amino acid” refers to an amino acid comprising hydrophobic properties e.g. alanine, cysteine, phenylalanine, glycine, histidine, isoleucine, lysine, leucine, methionine, arginine, threonine, valine, tryptophan, tyrosine and others listed in Table 3 hereinbelow.

As used herein, the phrase “small amino acid” refers to amino acids with a volume of Van der Waals (A³) that is from about 60-120 and including valine and its derivatives. Examples of such amino acids include, but are not limited to alanine, cysteine, aspartic acid, glycine, asparagine, proline, serine, threonine, valine and others listed in Table 3 hereinbelow.

As used herein, the phrase “turnlike amino acid” refers to an amino acid comprising a bendable bond. Examples of such amino acids include, but are not limited to alanine, cysteine, aspartic acid, glutamic acid, glycine, histidine, lysine, asparagine, glutamine, arginine, serine, threonine and others listed in Table 3 hereinbelow.

As used herein, the phrase “polar amino acid” refers to those amino acids with side-chains that prefer to reside in an aqueous (i.e. water) environment. Exemplary polar amino acids include but are not limited to cysteine, aspartic acid, glutamic acid, histidine, lysine, asparagine, glutamine, arginine, serine, threonine and others listed in Table 3 hereinbelow.

As used herein, the phrase “aromatic amino acid” refers to amino acids comprising an aromatic side chain (i.e. an aromatic ring system). Exemplary aromatic amino acids include but are not limited to glutamic acid, histidine, tryptophan, tyrosine and others listed in Table 3 hereinbelow.

According to another embodiment, the amino acid sequence of the OCLP1 polypeptides of the present invention confers about 80% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a hydrophobic amino acid, X₅ is glycine, X₆ is a polar amino acid, X₈ is cysteine, X₉ is a hydrophobic amino acid, X₁₀ is a turnlike amino acid, X₁ is a polar amino acid, X₁₂ is a turnlike amino acid, X₁₄ is a polar amino acid, X₁₅ is cysteine, X₁₆ is cysteine, X₁₇ is a small amino acid, X₂₀ is a turnlike amino acid, X₂₁ is a turnlike amino acid, X₂₂ is cysteine, X₂₃ is a hydrophobic amino acid, X₂₄ is a small amino acid, X₂₅ is an aromatic amino acid, X₂₆ is a turnlike amino acid, X₂₈ is a positive amino acid, X₂₉ is cysteine and X₃₀ is an aliphatic amino acid.

As used herein, the phrase “positive amino acid” refers to an amino acid comprising an overall positive charge at physiological pH, such as histidine, lysine or arginine and others referred to in Table 3 hereinbelow.

As used herein, the phrase “aliphatic amino acid” refers to amino acids comprising a protein side chain containing only carbon or hydrogen atoms. Methionine may also be considered in this category. Although its side-chain contains a sulphur atom, it is largely non-reactive, meaning that Methionine effectively susbsitutes well with the true aliphatic amino acaids. Other exemplary aliphatic amino acids include, but are not limited to isoleucine, leucine or valine and others listed in Table 3 hereinbelow.

According to yet another embodiment, the amino acid sequence of the OCLP1 polypeptides of the present invention confers about 70% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a small amino acid, X₃ is a turn-like amino acid, X₄ is a small amino acid, X₅ is glycine, X₆ is a polar amino acid, X₇ is a hydrophobic amino acid, X₈ is cysteine, X₉ is a hydrophobic amino acid, X₁₀ is a small amino acid, X₁₁ is a polar amino acid, X₁₂ is a small amino acid, X₁₄ is a polar amino acid, X₁₅ is cysteine, X₁₆ is cysteine, X₁₇ is serine, X₂₀ is a small amino acid, X₂₁ is a small amino acid, X₂₂ is cysteine, X₂₃ is a hydrophobic amino acid, X₂₄ is a small amino acid, X₂₅ is an aromatic amino acid, X₂₆ is a tiny amino acid, X₂₇ is a hydrophobic amino acid, X₂₈ is a positive amino acid, X₂₉ is cysteine and X₃₀ is valine.

As used herein, the phrase “tiny amino acid” refers to those amino acids with a volume of Van der Waals (A³) that is from about 60-90. Exemplary tiny amino acids include, but are not limited to alanine, glycine or serine and others listed in Table 3 hereinbelow.

According to still another embodiment, the amino acid sequence of the OCLP1 polypeptides of the present invention confers about 60% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

Where X₁ is cysteine, X₂ is a tiny amino acid, X₃ is a turn-like amino acid, X₄ is a small amino acid, X₅ is glycine, X₆ is a negative amino acid, X₇ is an aromatic amino acid, X₈ is cysteine, X₉ is an aliphatic amino acid, X₁₀ is a small amino acid, X₁ is a charged amino acid, X₁₂ is a small amino acid, X₁₄ is a negative amino acid, X₁₅ is cysteine, X₁₆ is cysteine, X₁₇ is serine, X₂₀ is a small amino acid, X₂₁ is a small amino acid, X₂₂ is cysteine, X₂₃ is leucine, X₂₄ is an alcoholic amino acid, X₂₅ is tyrosine, X₂₆ is a tiny amino acid, X₂₇ is a hydrophobic amino acid, X₂₈ is lysine, X₂₉ is cysteine and X₃₀ is valine.

As used herein, the phrase “negative amino acid” refers to an amino acid comprising an overall negative charge at physiological pH. Exemplary negative amino acids include, but are not limited to aspartic acid or glutamic acid and others listed in Table 3, hereinbelow.

As used herein, the phrase “alcoholic amino acid” refers to an amino acid comprising an OH group. Exemplary alcoholic amino acids include but are not limited to serine or threonine and others listed in Table 3 hereinbelow.

As used herein the phrase “charged amino acid” refers to an amino acid that carries an overall charge at physiological pH. Such amino acids include, but are nto limited to aspartic acid, glutamic acid, histidine, lysine or arginine and others listed in Table 3 hereinbelow.

According to still another embodiment, the amino acid sequence of the OCLP1 polypeptides of the present invention confers about 50% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a tiny amino acid, X₃ is a turn-like amino acid, X₄ is a small amino acid, X₅ is glycine, X₆ is Glutamic acid, X₇ is an aromatic amino acid, where X₈ is cysteine, X₉ is lysine, X₁₀ is an alcoholic amino acid, X₁₁ is histidine, X₁₂ is a small amino acid, X₁₄ is a negative amino acid, where X₁₅ is cysteine, where X₁₆ is cysteine, X₁₇ is serine, X₂₀ is a small amino acid, X₂₁ is a tiny amino acid, where X₂₂ is cysteine, X₂₃ is leucine, X₂₄ is an alcoholic amino acid, X₂₅ is tyrosine, X₂₆ is a tiny amino acid, X₂₇ is a turn-like amino acid, X₂₈ is lysine, where X₂₉ is cysteine and X₃₀ is valine.

According to still another embodiment, the amino acid sequence of the OCLP1 polypeptides of the present invention confers about 90% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₅ is glycine, X₆ is a turnlike amino acid, X₈ is cysteine, X₉ is a hydrophobic amino acid, X₁₀ is a turnlike amino acid, X₁₁ is a polar amino acid, X₁₂ is a turnlike amino acid, X₁₅ is cysteine, X₁₆ is cysteine, X₁₇ is a polar amino acid, X₂₂ is cysteine, X₂₅ is an aromatic amino acid, X₂₆ is an turnlike amino acid, X₂₈ is a polar amino acid and X₂₉ is cysteine.

According to still another embodiment, the amino acid sequence of the OCLP1 polypeptides of the present invention confers about 80% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a hydrophobic amino acid, X₅ is glycine, X₆ is a turnlike amino acid, X₈ is cysteine, X₉ is a hydrophobic amino acid, X₁₀ is a turnlike amino acid, X₁₁ is a polar amino acid, X₁₂ is a small amino acid, X₁₄ is a polar amino acid, X₁₅ is cysteine, X₁₆ is cysteine, X₁₇ is a small amino acid, X₂₀ is a turnlike amino acid, X₂₂ is cysteine, X₂₃ is a hydrophobic amino acid, X₂₅ is tyrosine, X₂₆ is a small amino acid, X₂₈ is a positive amino acid, X₂₉ is cysteine and X₃₀ is a hydrophobic amino acid.

According to still another embodiment, the amino acid sequence of the OCLP1 polypeptides of the present invention confers about 70% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a hydrophobic amino acid, X₃ is a small amino acid, X₄ is a hydrophobic amino acid, X₅ is glycine, X₆ is a polar amino acid, X₈ is cysteine, X₉ is a hydrophobic amino acid, X₁₀ is a turnlike amino acid, X₁₁ is a polar amino acid, X₁₂ is a small amino acid, X₁₄ is a polar amino acid, X₁₅ is cysteine, X₁₆ is cysteine, X₁₇ is a small amino acid, X₂₀ is a turnlike amino acid, X₂₁ is a turnlike amino acid, X₂₂ is cysteine, X₂₃ is a hydrophobic amino acid, X₂₄ is a small amino acid, X₂₅ is tyrosine, X₂₆ is a tiny amino acid, X₂₈ is a positive amino acid, X₂₉ is cysteine and X₃₀ is a small amino acid.

According to still another embodiment, the amino acid sequence of the OCLP1 polypeptides of the present invention confers about 60% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a turnlike amino acid, X₃ is a small amino acid, X₄ is a hydrophobic amino acid, X₅ is glycine, X₆ is a polar amino acid, X₈ is cysteine, X₉ is a hydrophobic amino acid, X₁₀ is a small amino acid, X₁₁ is a polar amino acid, X₁₂ is a small amino acid, X₁₄ is a polar amino acid, X₁₅ is cysteine, X₁₆ is cysteine, X₁₇ is a small amino acid, X₂₀ is a turnlike amino acid, X₂₁ is a polar amino acid, X₂₂ is cysteine, X₂₃ is a hydrophobic amino acid, X₂₄ is a small amino acid, X₂₅ is tyrosine, X₂₆ is a tiny amino acid, X₂₇ is a small amino acid, X₂₈ is a positive amino acid, X₂₉ is cysteine and X₃₀ is an aliphatic amino acid.

According to still another embodiment, the amino acid sequence of the OCLP1 polypeptides of the present invention confers about 50% to the consensus:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is cysteine, X₂ is a tiny amino acid, X₃ is a tiny amino acid, X₄ is a small amino acid, X₅ is glycine, X₆ is a negative amino acid, X₇ is a polar amino acid, X₈ is cysteine, X₉ is an aliphatic amino acid, X₁₀ is a small amino acid, X₁₁ is a small amino acid, X₁₂ is a small amino acid, X₁₄ is a negative amino acid, X₁₅ is cysteine, X₁₆ is cysteine, X₁₇ is serine, X₂₀ is a small amino acid, X₂₁ is a small amino acid, X₂₂ is cysteine, X₂₃ is a hydrophobic amino acid, X₂₄ is an alcoholic amino acid, X₂₅ is tyrosine, X₂₆ is alanine, X₂₇ is a small amino acid, X₂₈ is lysine, X₂₉ is cysteine and X₃₀ is valine.

As mentioned herein above, the polypeptides of the present invention may be modified. Such modifications include C terminus modification. The present inventors have shown that C terminal amidation is required for functionality. Other modifications include, but are not limited to N terminus modification, polypeptide bond modification, including, but not limited to, CH2-NH, CH2-S, CH2—S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 2 and 3 below list naturally occurring amino acids (Table 2) and non-conventional or modified amino acids (Table 3) which can be used with the present invention.

TABLE 2

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid | Xaa | X |
| as above | | |

TABLE 3

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α ethylhistidine | Mhis | L-α-methylhomophenylalanine | Mhphe |
| L-α thylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α ethylvaline | Mtrp | L-α-methyltyrosine | Mtyr |
| L-α-methylleucine | Mval [symbol] bhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| [symbol] ylamino)cyclopropane | | | |

[symbol]

indicates data missing or illegible when filed

The present invention also conceives of modifications which aid in the targeting of the polypeptides to a particular site in the body.

Thus, according to an embodiment of this aspect of the present invention, the polypeptides of the present invention may be attached to an affinity moiety, such as an antibody, a receptor ligand or a carbohydrate to generate targeting molecules. Examples of antibodies which may be used according to this aspect of the present invention include but are not limited to tumor antibodies, anti CD20 antibodies and anti-IL 2R alpha antibodies. Exemplary receptors include, but are not limited to folate receptors and EGF receptors. An exemplary carbohydrate which may be used according to this aspect of the present invention is lectin. Since, it is expected that the polypeptides of the present invention may comprise toxic like properties (i.e. comprise cytotoxic activity), the polypeptides may be useful in killing cells. Thus, the target cells may be metastasized cancer cells expressing identifiable surface markers.

The affinity moiety may be covalently or non-covalently linked to or adsorbed on to the polypeptides of the present invention using any linking or binding method and/or any suitable chemical linker known in the art. The exact type and chemical nature of such cross-linkers and cross linking methods is preferably adapted to the type of affinity group used and the exact sequence of the polypeptide of the present invention. Methods for binding or adsorbing or linking such affinity labels and groups are also well known in the art.

Since the isolated polypeptides of the present invention typically comprise about 25-30 amino acids, they can be biochemically synthesized such as by using standard solid phase techniques. These methods include exclusive solid phase synthesis, partial solid phase synthesis methods, fragment condensation, classical solution synthesis.

Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

Alternatively, the polypeptides of the present invention may be isolated from the secretion glands of the appropriate insect using methods known in the art such as affinity isolation using an appropriate antibody or any other peptide separation procedure.

Recombinant techniques may also be used to generate the isolated polypeptides of the present invention. This may be particularly appropriate when generation of large amounts of the polypeptides are required. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

These techniques may be used to generate the polypeptide of the present invention in vitro, ex vivo and in vivo (the latter two are further described hereinbelow).

To produce the isolated OCLP1 polypeptides of the present invention using recombinant technology, an isolated polynucleotide comprising a nucleic acid sequence encoding such a polypeptide may be used. Exemplary nucleic acid sequences are set forth in SEQ ID NOs: 13 and 14. Exemplary nucleic acid sequences encoding the OCLP1 ortholog polypeptides of the present invention are set forth in SEQ ID NOs: 15-19.

The term “nucleic acid sequence” refers to a deoxyribonucleic acid sequence composed of naturally-occurring bases, sugars and covalent internucleoside linkages (e.g., backbone) as well as oligonucleotides having non-naturally-occurring portions which function similarly to respective naturally-occurring portions. Such modifications are enabled by the present invention provided that recombinant expression is still allowed.

A nucleic acid sequence of OCLP1 according to this aspect of the present invention can be a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

In order to generate the OCLP1 polypeptides of the present invention using recombinant techniques, the polynucleotides encoding same are ligated into nucleic acid expression vectors, such that the polynucleotide sequence is under the transcriptional control of a cis-regulatory sequence (e.g., promoter sequence).

A variety of prokaryotic or eukaryotic cells can be used as host-expression systems to express the polypeptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

Constitutive promoters suitable for use with this embodiment of the present invention include sequences which are functional (i.e., capable of directing transcription) under most environmental conditions and most types of cells such as the cytomegalovirus (CMV) and Rous sarcoma virus (RSV).

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide. For example, the present inventors expressed active OCLP1 in bacteria with a cellulose tag to aid in purification which was later cleaved prior to use (see Example 3 of the Examples section hereinbelow).

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is effected.

The phrase “recovering the recombinant polypeptide” used herein refers to collecting the whole fermentation medium containing the polypeptide and need not imply additional steps of separation or purification.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

As mentioned hereinabove, the polypeptides of the present invention may be expressed in vivo or ex vivo (i.e. using gene therapy techniques).

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRepS, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

According to one embodiment of this aspect of the present invention, inducible promoters may be used for gene therapy. Accordingly, the polypeptides of the present invention may be up-regulated during acute phases of a chronic disease (e.g. cancer) or pain. An example of such an inducible promoter is the tetracycline-inducible promoter (Srour, M. A., et al., 2003. Thromb. Haemost. 90: 398-405).

It will be appreciated that using the bioinformatics method of the present invention, the present inventors identified other novel toxin like polypeptides.

Thus, the present invention encompasses polypeptides comprising an amino acid sequence as set forth in SEQ ID NO: 35, also referred to herein as raalin, its orthologs comprising amino acid sequences as set forth in SEQ ID NOs: 31-34 and homologs, active fragments, derivatives and modified forms thereof. According to an embodiment of this aspect of the present invention, the raalin polypeptides conform about 70% to the consensus sequence:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀

where X₁ is a big amino acid, X₃ is cysteine, X₄ is aspartic acid, X₅ is serine or threonine, X₈ is a positive amino acid, X₉ is glutamic acid, X₁₁ is a small amino acid, X₁₂ is a small amino acid, X₁₃ is alanine, X₁₄ is a negative amino acid, X₁₇ is a polar amino acid, X₁₈ is histidine, X₂₀ is arginine, X₂₁ is serine or threonine, X₂₆ is tyrosine, X₂₇ is an aliphatic amino acid, X₂₈ is a positive amino acid, X₂₉ is a positive amino acid and X₃₀ is a positive amino acid.

As used herein, the phrase “big amino acid” refers to amino acids with a volume of Van der Waals (A³) that is from about 120 or more including, but not limited to glutamic acid, phenylalanine, histidine, isoleucine, leucine, methionine, glutamine, arginine, tryptophan or tyrosine and other derivatives listed in Table 3 hereinabove.

Furthermore, the present invention encompasses the isolated polynucleotides encoding the above mentioned polypeptides comprising nucleic acid sequences e.g. as set forth in SEQ ID NOs: 36-38 and cells expressing same.

Other polypeptides identified by the bioinformatics method of the present invention include mouse polypeptides comprising amino acid sequences as set forth in SEQ ID NOs: 39-46 having nucleic acid sequences encoding same as set forth in SEQ ID NOs: 47-56 and human polypeptides comprising amino acid sequences as set forth in SEQ ID NOs: 57-59 having nucleic acid sequences encoding same as set forth in SEQ ID NOs: 60-62.

It will be appreciated that the present inventors identified consensus sequences for the above mentioned mouse and human polypeptides. Thus the present invention also includes other polypeptides which conform to the consensus sequences hereinbelow. Thus, for example, the present invention incorporates all polypeptides that conform at least 90% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

where X₅ is a hydrophobic amino acid, X₇ is cysteine, X₁₀ is cysteine, X₁₁ is a turnlike amino acid, X₁₅ is a polar amino acid, X₁₈ is a hydrophobic amino acid, X₁₉ is cysteine, X₂₄ is a turnlike amino acid, X₂₆ is cysteine, X₂₈ is a hydrophobic amino acid, X₂₉ is a polar amino acid, X₃₅ is a turnlike amino acid, X₃₆ is a polar amino acid, X₃₈ is cysteine, X₄₀ is a hydrophobic amino acid, X₄₃ is a hydrophobic amino acid, X₄₄ is an aromatic amino acid, X₄₇ is a small amino acid, X₄₈ is a charged amino acid, X₅₅ is a hydrophobic amino acid and X₅₇ is a hydrophobic amino acid.

Furthermore, the present invention incorporates all polypeptides that conform at least 80% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₄ is a hydrophobic amino acid, X₅ is an aliphatic amino acid, X₇ is cysteine, X₈ is a hydrophobic amino acid, X₉ is a polar amino acid, X₁₀ is cysteine, X₁₁ is a turnlike amino acid, X₁₂ is a hydrophobic amino acid, X₁₅ is a polar amino acid, X₁₆ is a turnlike amino acid, X₁₇ is a tiny amino acid, X₁₈ is a hydrophobic amino acid, X₁₉ is cysteine, X₂₀ is a hydrophobic amino acid, X₂₁ is a turnlike amino acid, X₂₂ is a small amino acid, X₂₃ is a polar amino acid, X₂₄ is a small amino acid, X₂₅ is a small amino acid, X₂₆ is cysteine, X₂₈ is a small amino acid, X₂₉ is a polar amino acid, X₃₅ is a polar amino acid, X₃₆ is a polar amino acid, X₃₇ is a turnlike amino acid, X₃₈ is cysteine, X₃₉ is a hydrophobic amino acid, X₄₀ is a hydrophobic amino acid, X₄₁ is a turnlike amino acid, X₄₂ is a turnlike amino acid, X₄₃ is a hydrophobic amino acid, X₄₄ is an aromatic amino acid, X₄₆ is a hydrophobic amino acid, X₄₇ is a small amino acid, X₄₈ is a positive amino acid, X₅₅ is a hydrophobic amino acid, X₅₇ is an aromatic amino acid and X₅₉ is a hydrophobic amino acid.

Furthermore, the present invention incorporates all polypeptides that conform atleast70% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₄ is a hydrophobic amino acid, X₅ is leucine, X₆ is a polar amino acid, X₇ is cysteine, X₈ is a hydrophobic amino acid, X₉ is a polar amino acid, X₁₀ is cysteine, X₁₁ is a turnlike amino acid, X₁₂ is a hydrophobic amino acid, X₁₅ is a charged amino acid, X₁₆ is a turnlike amino acid, X₁₇ is a tiny amino acid, X₁₈ is a charged amino acid, X₁₉ is cysteine, X₂₀ is a hydrophobic amino acid, X₂₁ is a turnlike amino acid, X₂₂ is a small amino acid, X₂₃ is a charged polar amino acid, X₂₄ is a small amino acid, X₂₅ is a small amino acid, X₂₆ is cysteine, X₂₇ is a hydrophobic amino acid, X₂₈ is a small amino acid, X₂₉ is a polar amino acid, X₃₄ is a polar amino acid, X₃₅ is a small amino acid, X₃₆ is a polar amino acid, X₃₇ is a polar amino acid, X₃₈ is cysteine, X₃₉ is a hydrophobic amino acid, X₄₀ is a hydrophobic amino acid, X₄₁ is a polar amino acid, X₄₂ is a polar amino acid, X₄₃ is a hydrophobic amino acid, X₄₄ is an aromatic amino acid, X₄₆ is a turnlike amino acid, X₄₇ is a small amino acid, X₄₉ is a positive amino acid, X₅₅ is a hydrophobic amino acid, X₅₆ is a polar amino acid, X₅₇ is an aromatic amino acid, X₅₈ is a hydrophobic amino acid and X₅₉ is a hydrophobic amino acid.

Furthermore, the present invention incorporates all polypeptides that conform at least 60% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₄ is a hydrophobic amino acid, X₅ is leucine, X₆ is a polar amino acid, X₇ is cysteine, X₈ is a hydrophobic amino acid, X₉ is a small amino acid, X₁₀ is cysteine, X₁₁ is a turnlike amino acid, X₁₂ is a hydrophobic amino acid, X₁₄ is a small amino acid, X₁₅ is a charged amino acid, X₁₆ is apolar amino acid, X₁₇ is Glycine, X₁₈ is a positive amino acid, X₁₉ is cysteine, X₂₀ is a hydrophobic amino acid, X₂₁ is a polar amino acid, X₂₂ is Glycine, X₂₃ is a charged polar amino acid, X₂₄ is a small amino acid, X₂₅ is an alcoholic amino acid, X₂₆ is cysteine, X₂₇ is a hydrophobic amino acid, X₂₈ is a small amino acid, X₂₉ is a polar amino acid, X₃₄ is a small amino acid, X₃₅ is a small amino acid, X₃₆ is a polar amino acid, X₃₇ is a polar amino acid, X₃₈ is cysteine, X₃₉ is a hydrophobic amino acid, X₄₀ is an aliphatic amino acid, X₄, is a charged amino acid, X₄₂ is a polar amino acid, X₄₃ is a hydrophobic amino acid, X₄₄ is phenylalanine, X₄₅ is a charged amino acid, X₄₆ is a small amino acid, X₄₇ is a small amino acid, X₄₈ is lysine, X₅₅ is a hydrophobic amino acid, X₅₆ is a polar amino acid, X₅₇ is an aromatic amino acid, X₅₈ is a small amino acid, X₅₉ is a hydrophobic amino acid and X₆₀ is a polar amino acid.

Furthermore, the present invention incorporates all polypeptides that conform at least 50% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₃ is a hydrophobic amino acid, X₄ is a small amino acid, X₅ is leucine, X₆ is a small amino acid, X₇ is cysteine, X₈ is an aromatic amino acid, X₉ is an alcoholic amino acid, X₁₀ is cysteine, X₁₁ is a small amino acid, X₁₂ is a polar amino acid, X₁₃ is a hydrophobic amino acid, X₁₄ is Asparagine, X₁₅ is a charged amino acid, X₁₆ is a small amino acid, X₁₇ is Glycine, X₁₈ is Lysine, X₁₉ is cysteine, X₂₀ is a hydrophobic amino acid, X₂₁ is a small amino acid, X₂₂ is Glycine, X₂₃ is glutamic acid, X₂₄ is glycine, X₂₅ is an alcoholic amino acid, X₂₆ is cysteine, X₂₇ is a polar amino acid, X₂₈ is threonine, X₂₉ is a polar amino acid, X₃₄ is a small amino acid, X₃₅ is a tiny amino acid, X₃₆ is a charged amino acid, X₃₇ is a small amino acid, X₃₈ is cysteine, X₃₉ is a small amino acid, X₄₀ is an aliphatic amino acid, X₄ is a positive amino acid, X₄₂ is a polar amino acid, X₄₃ is a hydrophobic amino acid, X₄₄ is phenylalanine, X₄₅ is a charged amino acid, X₄₆ is glycine, X₄₇ is glycine, X₄₈ is lysine, X₅₅ is an aromatic amino acid, X₅₆ is glutamine, X₅₇ is an aromatic amino acid, X₅₈ is a tiny amino acid, X₅₉ is a polar amino acid and X₆₀ is glutamine.

Furthermore, the present invention incorporates all polypeptides that conform at least 90% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₂ is cysteine, X₆ is cysteine, X₈ is a turn-like amino acid, X₁₀ is a polar amino acid, X₁₇ is a hydrophobic amino acid, X₁₈ is a hydrophobic amino acid, X₁₉ is a hydrophobic amino acid, X₂₁ is a hydrophobic amino acid, X₂₂ is a hydrophobic amino acid, X₂₃ is cysteine, X₂₄ is cysteine, X₂₇ is a polar amino acid, X₂₈ is a polar amino acid, X₂₉ is a small amino acid, X₃₀ is a hydrophobic amino acid, X₃₁ is cysteine and X₃₂ is asparagine.

Furthermore, the present invention incorporates all polypeptides that conform at least 80% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₁ is a turn-like amino acid, X₂ is cysteine, X₄ is a turn-like amino acid, X₆ is cysteine, X₈ is a small amino acid, X₁₀ is a polar amino acid, X₁₂ is a hydrophobic amino acid, X₁₅ is a turn-like amino acid, X₁₆ is a small amino acid, X₁₇ is a hydrophobic amino acid, X₁₈ is a hydrophobic amino acid, X₁₉ is a hydrophobic amino acid, X₂₀ is a polar amino acid, X₂₁ is a hydrophobic amino acid, X₂₂ is a hydrophobic amino acid, X₂₃ is cysteine, X₂₄ is cysteine, X₂₆ is a polar amino acid, X₂₇ is a polar amino acid, X₂₈ is a polar amino acid, X₂₉ is a small amino acid, X₃₀ is a hydrophobic amino acid, X₃₁ is cysteine, X₃₂ is asparagines and X₃₃ is a polar amino acid.

Furthermore, the present invention incorporates all polypeptides that conform at least 70% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₁ is a turn-like amino acid, X₂ is cysteine, X₃ is a turn-like amino acid, X₄ is a small amino acid, X₆ is cysteine, X₈ is a small amino acid, X₉ is a hydrophobic amino acid, X₁₀ is a polar amino acid, X₁₂ is a hydrophobic amino acid, X₁₃ is a hydrophobic amino acid, X₁₄ is a turn-like amino acid, X₁₅ is a turn-like amino acid, X₁₆ is a small amino acid, X₁₇ is a hydrophobic amino acid, X₁₈ is a hydrophobic amino acid, X₁₉ is a hydrophobic amino acid, X₂₀ is a polar amino acid, X₂₁ is a hydrophobic amino acid, X₂₂ is a hydrophobic amino acid, X₂₃ is cysteine, X₂₄ is cysteine, X₂₆ is a polar amino acid, X₂₇ is a polar amino acid, X₂₈ is a polar amino acid, X₂₉ is a small amino acid, X₃₀ is an aromatic amino acid, X₃₁ is cysteine, X₃₂ is asparagines, X₃₃ is a charged amino acid and X₃₄ is a hydrophobic amino acid.

Furthermore, the present invention incorporates all polypeptides that conform at least 60% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₁ is a small amino acid, X₂ is cysteine, X₃ is a polar amino acid, X₄ is a small amino acid, X₅ is a turn-like amino acid, X₆ is cysteine, X₈ is a small amino acid, X₉ is a hydrophobic amino acid, X₁₀ is a small amino acid, X₁₂ is a hydrophobic amino acid, X₁₃ is a hydrophobic amino acid, X₁₄ is a small amino acid, X₁₅ is a polar amino acid, X₁₆ is a small amino acid, X₁₇ is a polar amino acid, X₁₈ is a polar amino acid, X₁₉ is a hydrophobic amino acid, X₂₀ is a polar amino acid, X₂₁ is a hydrophobic amino acid, X₂₂ is a hydrophobic amino acid, X₂₃ is cysteine, X₂₄ is cysteine, X₂₆ is a charged amino acid, X₂₇ is a small amino acid, X₂₈ is a polar amino acid, X₂₉ is a small amino acid, X₃₀ is an aromatic amino acid, X₃₁ is cysteine, X₃₂ is asparagines, X₃₃ is a charged amino acid and X₃₄ is a hydrophobic amino acid.

Furthermore, the present invention incorporates all polypeptides that conform at least 50% to:

X₁X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅X₂₆X₂₇X₂₈X₂₉X₃₀X₃₁X₃₂X₃₃X₃₄X₃₅
X₃₆X₃₇X₃₈X₃₉X₄₀X₄₁X₄₂X₄₃₁X₄₄X₄₅X₄₆X₄₇X₄₈X₄₉X₅₀X₅₁
X₅₂X₅₃X₅₄X₅₅X₅₆X₅₇X₅₈X₅₉X₆₀

Where X₁ is a tiny amino acid, X₂ is cysteine, X₃ is a charged amino acid, X₄ is a small amino acid, X₅ is a small amino acid, X₆ is cysteine, X₇ is a small amino acid, X₈ is a small amino acid, X₉ is a hydrophobic amino acid, X₁₀ is asparagine, X₁₂ is a an aliphatic amino acid, X₁₃ is a hydrophobic amino acid, X₁₄ is an alcoholic amino acid, X₁₅ is a positive amino acid, X₁₆ is a small amino acid, X₁₇ is a polar amino acid, X₁₈ is arginine, X₁₉ is a hydrophobic amino acid, X₂₀ is a polar amino acid, X₂₁ is an aliphatic amino acid, X₂₂ is a hydrophobic amino acid, X₂₃ is cysteine, X₂₄ is cysteine, X₂₆ is a positive amino acid, X₂₇ is a charged amino acid, X₂₈ is a charged amino acid, X₂₉ is a small amino acid, X₃₀ is phenylalanin, X₃₁ is cysteine, X₃₂ is asparagine, X₃₃ is lysine and X₃₄ is a hydrophobic amino acid.

As mentioned hereinabove, the present inventors have shown that the polypeptides of the present invention (e.g. active OCLP1) exert a biological effect on vertebrates (a reversible paralysis in fish). Furthermore, OCLP1 injection into Xenopus oocytes previously transfected with ion channels known to be associated with pain (Ca channel α₁, α₂, and β subunits), caused a consistent change of 10% in current flow, indicating that OCLP1 may have an effect on pain (FIGS. 11A-D). In addition OCLP1 possesses a fold similar to that of ω-conotoxin (a toxin known to comprise analgesic activities) as determined by the PHYRE fold recognition server.

Accordingly, the present inventors propose that the polypeptides of the present invention may be used for treating a nerve disease or disorder. The method comprises administering to a subject in need thereof a therapeutically effective amount of the polypeptides of the present invention.

As used herein the term “treating” refers to preventing, alleviating or diminishing a symptom associated with a nerve disease or disorder. Preferably, treating cures, e.g., substantially eliminates, the symptoms associated with the nerve disease or disorder.

As used herein the term “subject” refers to any (e.g., mammalian) subject, preferably a human subject.

The phrase “nerve disease or disorder” as used herein refers to any medical condition which is accompanied by neurological symptoms and thus includes both CNS diseases or disorders and peripheral nerve diseases or disorders.

Examples of CNS diseases or disorders include but are not limited to a pain disorder, a motion disorder, a dissociative disorder, a mood disorder, an affective disorder, a neurodegenerative disease or disorder, an addictive disorder and a convulsive disorder.

For example, the CNS disease or disorder may be Parkinson's, Multiple Sclerosis, Huntington's disease, action tremors and tardive dyskinesia, panic, anxiety, depression, Alzheimer's or epilepsy.

Exemplary peripheral nerve diseases or disorders include hereditary neuropathy, a mononeuritis multiplex, a mononeuropathy, a muscle stimulation disorder, a neuromuscular junction disorder, a plexus disorder, a polyneuropathy, a spinal muscular atrophy and a thoracic outlet syndrome.

The advantage of using venom peptides and toxin like proteins such as the OCLP1 polypeptides of the present invention as therapeutic agents, resides in the fact that they are poorly immunogenic when injected in the absence of an adjuvant (Maillere et al., J. Immunol. 1993 Jun. 15; 150(12):5270-80). In addition the toxins' high potency allows them to be used in minute amounts, so that production costs may not be a limiting factor. Furthermore the toxins' high specificity reduces the risk of adverse reactions. In addition, unlike most small-molecule based drugs, toxins degrade into amino acids, reducing the risk of metabolite toxicity.

The polypeptides of the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the toxin like polypeptides accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As mentioned hereinabove, one biological activity identified with the polypeptides of the present invention was the ability to paralyse muscles in a fish. One feature of botulinium toxin, a well known toxin, is its ability to paralyse the corrugator and procerus muscles. This feature is exploited for the treatment of galbellar frown lines (wrinkles). Since the polypeptides of the present invention were identified as comprising toxin-like features, the present inventors propose that these polypeptides may, in a similar way to botulinium toxin (botox Tm) be useful in a cosmetic preparation (e.g., injectable) for the treatment of wrinkles.

Toxins that are capable of inhibiting insect Ca channels are known to comprise insecticidal activities (see e.g. U.S. Pat. Appl. No. 20030199039). Since the polypeptides of the present invention were identified on the basis that they comprise structural features similar to ion channel inhibitors, the present inventors envisage that they may be used for controlling or exterminating pests such as insects. The method comprises applying to the insect or crop an insecticidally effective amount of the isolated polypeptides of the present invention.

Crops for which this approach would be useful are numerous, including, but not limited to, cotton, tomato, green bean, sweet corn, lucerne, soybean, sorghum, field pea, linseed, safflower, rapeseed, sunflower, and field lupins.

Insect infestation of crops may be controlled by treating the crops and/or insects with such compositions. The insects and/or their larvae may be treated with the composition, for example, by attracting the insects to the composition with an attractant.

The formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art.

The term “agriculturally-acceptable carrier” covers all adjuvants, inert components, dispersants, surfactants, tackifiers, binders, etc. that are ordinarily used in pesticide formulation technology; these are well known to those skilled in pesticide formulation. The formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in U.S. Pat. No. 6,468,523, herein incorporated by reference.

The term “pest” as used herein, includes but is not limited to, insects, fungi, bacteria, nematodes, mites, ticks, and the like. Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Coleoptera, Lepidoptera, and Diptera.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize billbug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafmniner; Anaphothrips obscrurus, grass thrips; Solenopsis milesta, thief ant; Tetranychus urticae, two spotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, two spotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

Nematodes include parasitic nematodes such as root-knot, cyst, and lesion nematodes, including Heterodera spp., Meloidogyne spp., and Globodera spp.; particularly members of the cyst nematodes, including, but not limited to, Heterodera glycines (soybean cyst nematode); Heterodera schachtii (beet cyst nematode); Heterodera avenae (cereal cyst nematode); and Globodera rostochiensis and Globodera pailida (potato cyst nematodes). Lesion nematodes include Pratylenchus spp.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Construction of Toxin Protein Classifier

In light of the great diversity of toxins, it seems unfeasible to find a global characterization of toxins by direct sequence-based methods, as these proteins are not even alignable. However, in spite of their diversity, many toxins do share a common structural feature—a toxin-like stability (TLS). In many toxins, a relatively large number of disulfide bridges helps maintain rigid backbones, conferring high stability [Bastolla U, Demetrius L (2005) Protein Eng Des Sel 18: 405-415]. This property, in conjunction with other post-translational modifications such as glycosylation and amino acid modification [Craig A G, et al. (1998) Biochemistry 37:16019-16025; Craig AG, (1999) Eur J Biochem 264: 271-275], is hypothesized to help maintain the toxin's function while traveling through the recipient's hostile bloodstream.

Feature construction: The following 545 sequence-derived features were used to transform a given sequence into a vector
(I) Amino acid frequencies (20 features).
(II) Amino acid pair frequencies (400 features).
(III) Sequence length. Hereby referred to as m (1 feature).
(IV) Cysteine binary 5-mers (32 features). Sequence was divided into m−4 amino acid 5-mers. Each 5-mer was translated into a binary 5-mer. Cysteines were translated into 1, and the rest of the amino acids were translated into 0.
(V) Polarity binary 5-mers (32 features). Same as in (IV), except that Asp, Glu, Lys, Arg, Asn, Gln were translated into 1 and the rest of the amino acids were translated into 0.
(VI) Amino acid entropy (20 features). A quantitative measure of how each amino acid type is spread in the sequence. For a given amino acid type c, p₁, . . . , pₖ is marked, its positions in the sequence. Definitions: p₀=0 and pₖ₊₁=m+1; entropy of c is

$$entropy ( c ) = - {\sum }_{i = 1}^{k + 1} ( \frac{{p}_{i} - {p}_{i - 1}}{m} ) {log}_{2} ( \frac{{p}_{i} - {p}_{i - 1}}{m} ) .$$

(VII) Circular mean (40 features). A quantitative measure that encodes the relative location and spread of each amino acid type in the sequence. For a given amino acid type c, its positions are marked in the sequence by p₁, . . . , pₖ. The feature formalizes the following notion: If the sequence is spread clockwise around the 2-dimensional unit circle, the mean of the points on the circle can be calculated that match p₁, . . . , pₖ and defined as the circular mean of c. Formally, CM(c) may be defined as follows: CM(c)=(−2,2) if k=0 and

$$CM ( c ) = ( \frac{1}{k} {\sum }_{i = 1}^{k} sin ( \frac{2 π ( {p}_{i} - 1 )}{m} ) , \frac{1}{k} {\sum }_{i = 1}^{k} cos ( \frac{2 π ( {p}_{i} - 1 )}{m} ) )$$

otherwise.

Training set: To construct the training set, all sequences of proteins annotated in UniProt as ‘Ionic channel inhibitor’ were obtained. Fragments and proteins longer than 100 amino acids were excluded, leaving 534 ICI sequences. Note that this includes both mature peptides and preproteins. Next, clustering was performed in order to remove redundancy (necessary in order to avoid bias of the cross-validation results). Following this step, 289 proteins remained so that no two proteins share an identity of 80% or more. These proteins constitute the true training instances (the rationale for using only ICIs as true instances is discussed in the Results section). As for the false instances, these were randomly selected from UniProt. The false instances were generated in three sets: (I) Random full-length proteins; (II) Random fragments of random proteins, with lengths matching those of the true instances; (III) N-terminal fragments of random proteins, with lengths matching those of the true instances. The protein fragments are intended to avoid length bias, and the random fragments are intended to avoid N-terminal bias. Each of the three sets is twice the size of the set of true instances, a total of 1734 false instances. Following this, clustering is performed to remove redundancy (80% identity). The final training set consists of the union of the false and true non-redundant sets. Note that for each boosted stumps classifier, a separate false set is generated.

It is important to note that for prediction on the honey bee proteins, the sequences of apamin and MCDP (and their homologs) were not included in the training set.

Learning algorithm: The learning algorithm that was used is a meta-classifier based on the boosted stumps algorithm. A decision-stump is a decision-tree that has only one node. The stump classifier finds the best linear separation available by a single feature. In the boosted stumps method, the AdaBoost boosting algorithm [Feund Y, Schapire R E, Journal of Computer and System Sciences 1997, 55(1):119-139] is applied to the stump classifier. In order to determine the optimal number of iterations, a parameter-tuning framework was constructed in which, for a given parameter value, the classifier is evaluated by its AUC performance in a 3-fold cross validation test, and the parameter value which maximizes the AUC is chosen for the final classifier.

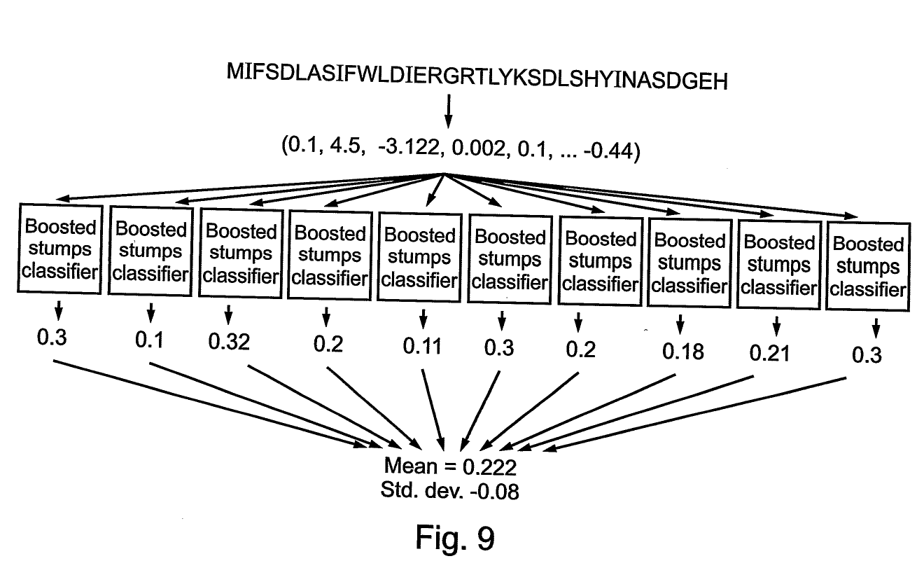

Classification of APTs is slightly different from the classical classification problem in the sense that a non well-defined property is being captured. Therefore, it was not clear that training the classifier to fit the training set well would translate into proper generalization, since some small portion of the labels is incorrect. Although some classifiers including AdaBoost are considered relatively resistant to label noise, an additional precaution was taken by constructing a meta-classifier as follows: For a given set of true instances, 10 sets of false instances were randomly generated (as described in “Training set”). Next, for each set of false instances a parameter-tuned boosted stump classifier was trained. The outputs of all 10 classifiers were normalized by the highest positive prediction of each classifier on the training set (respective to each classifier). The prediction of the meta-classifier is the mean average of the predictions of all 10 classifiers. Additionally, the meta-classifier provides the standard deviation of the predictions on each sequence as a measure of robustness. A prediction to be a positive prediction (i.e. the protein is APT) was considered if the mean was greater than the standard-deviation. By employing this meta-classifier approach a robust hypothesis was provided, which was not biased by any specific set of false instances. Note that in contrast to a classical classification scenario in which the whole training set (which includes all false instances) is fitted as best as possible and therefore possibly err on mislabeled instances, in the present method the chance of making a mistake on a specific mislabeled false instance is reduced, since that would require the false instance to be repeatedly chosen for the random false sets of the 10 sub-classifiers. An overview of the prediction procedure is shown in FIG. 9.

Sources and tools: All training set proteins were obtained from the UniProt database. The set of 29554 SwissProt proteins was obtained by taking all SwissProt proteins shorter than or equal to 150 aa and removing redundancy, so that following the process no two proteins are more than 90% identical. The set of 10157 honey bee predicted protein sequences is the official GLEAN3 predicted gene set (Gibbs et al., 2006). The set of 5154 novel mouse proteins was obtained from the website of the FANTOM project [Carninci et al., Science 2005, 309(5740):1559-1563]. SignalP [Bendsten et al., J Mol Biol 2004, 340(4):783-795] was used for predicting signal peptides. ClustalW [Thompson et al., Nucleic Acids Res 1994, 22(22):4673-4680] was used for multiple sequence alignment and phylogenetic analysis. NCBI-BLAST [Altschul et al., Nucleic Acids Res 1997, 25(17):3389-3402] was used for local alignment searches. PHYRE [Kelley et al, J Mol Biol 2000, 299(2):499-520] was used for fold recognition. InterProScan [Quevillon et al., Nucleic Acids Res 2005, 33 (Web Server issue):W116-120] was used for detection of sequence motifs. SDPMOD [Kong et al., Nucleic Acids Res 2004, 32 (Web Server issue):W356-359], a homology modeling tool that specializes in structures of small disulfide-rich proteins, was used to construct a 3D model of OCLP1. The ENSEMBL [Bimey et al., Nucleic Acids Res 2006, 34 (Database issue):D556-561] browser was used for genomic searches in Apis mellifera, Drosophila melanogaster, Anopheles gambiae and Aedes aegyptis. CD-HIT [Li et al., Bioinformatics 2002, 18(1):77-82] was used to cluster the sequences in order to construct non-redundant sets. All expression data was obtained from NCBI nucleotide and EST databases [Boguski et al, Nat Genet. 1993, 4(4):332-333]. Tribolium castaneum genomic search was performed in the Harvard Genome Sequencing Center website [Tribolium castaneum sequencing project]. The group designated ‘Antibacterial’ contains proteins that have at least one of the following UniProt keywords: ‘Antimicrobial’, ‘Fungicide’ and ‘Antibiotic’. The group designated ‘Venom proteins’ contains proteins whose UniProt entries stated localized expression in venom under the TISSUE field. ‘Snake toxin’, ‘Gonadotropin’, ‘Beta defensin’, ‘E6’ and ‘L36’ represent InterPro [Mulder et al, Nucleic Acids Res 2005, 33(Database issue):D201-205] groups IPRO03571, IPRO01545, IPRO01855, IPRO01334 and IPRO00473, respectively.

Results

A computational classifier was trained on a set of known ion channel inhibitors (ICIs) as described in Materials and Methods. ICIs are only a subset of all APTs. The reason ICIs were used for training rather than APTs is that the definition of structurally stable APTs (or APT-like proteins) is often confusing. For example, many proteins annotated as toxins (bacterial toxins, for example) may not naturally belong to this category. Furthermore, a bias from manual selection of the instances in the training set was avoided. Thus, the classifier was trained on the set of annotated ICIs with the hope that the classifier will generalize to include additional groups of APTs. This expectation is reasonable since ICIs by themselves are extremely variable in sequence, structure and function and are not known to share any ICI-specific features.

Most state-of-the-art functional classification methods use position specific information (e.g. evolutionary conserved positions) in order to find sequence motifs that are common to functional groups. Due to the large variation of APTs in sequence and structure, this commonly used approach is unsuitable in the case of APTs. The present classifier used 545 general sequence-derived features which were speculated to possibly be related to APT structural stability. The features were constructed so that they would reflect the frequency, distribution, packing and crude localization of cysteines within the sequence. However, the features were not restricted to cysteine-related features and were applied to all 20 amino-acids. See Methods section for a full description of the features.

The classifier was evaluated by a 3-fold cross-validation classification test. Area Under Curve (AUC) is an established measure of performance in this test, with AUC=1 indicating perfect success. The classifier obtained a mean AUC of 0.9934 (standard deviation=0.0026). The high performance in the cross-validation tests suggests that the classifier is indeed able to capture a robust phenomenon.

Although the classifier performs well on the cross-validation test, it is important to characterize what exactly the classifier has learned. For example, since the training set contained only ICIs as positive instances, the classifier was assessed as to whether it could detect only ICIs or other unrelated APT or APT-like groups as well. Generally, it would be a mistake to interpret the classifier's hypothesis as an explanation of an observed phenomenon. This is due to the fact that there is no preliminary reason that the characterization which the classifier has produced will be related in any way to a specific phenomenon. However, there is some indication that the present classifier's hypothesis is related to cysteine-mediated structural stability: Amongst all 545 sequence-derived features, the classifier repeatedly identified the most dominant feature to be the frequency of cysteines within the sequence.

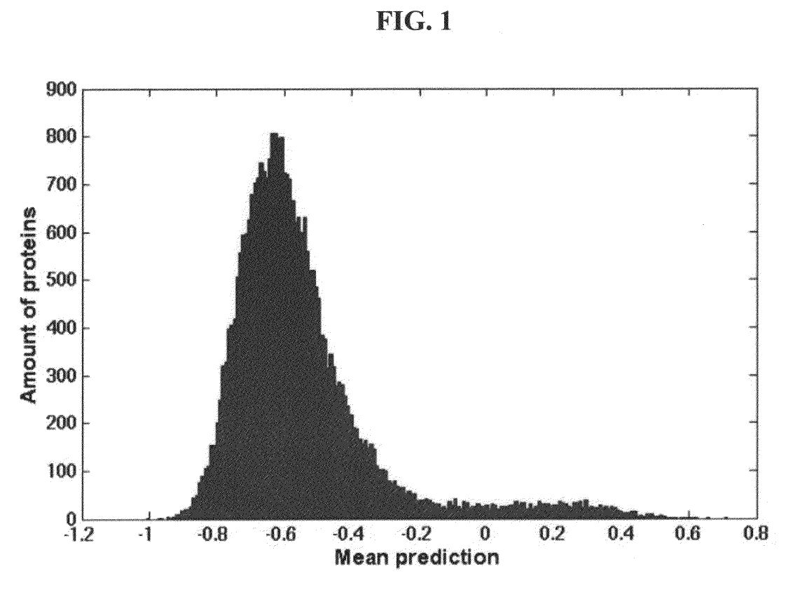

In order to assess the predictions made by the classifier, the classifier was applied to a non-redundant set of all 29554 SwissProt proteins shorter than or equal to 150 aa (excluding the ICIs which were present in the training set). A histogram of the predictions is shown in FIG. 1. 997 proteins (3.37%) were predicted positive by the classifier. In order to assess whether these were false positive predictions, the set of positive predictions was tested for enrichment in biological functional categories. For biological functional categories, the manually-validated UniProt keyword annotations was used and the predicted InterPro motif groups associate with the proteins. The results (Table 4, hereinbelow) show that the most highly over-represented groups were APT-related. Table 4 summarizes the statistically-enriched groups amongst the positive predictions.

TABLE 4

| Biological group* | Positive | Total | P-value |
|---|---|---|---|
| Toxin | 299 | 541 | 6.72E−303 |
| Neurotoxin | 172 | 242 | 2.896E−197 |
| Snake toxin | 119 | 137 | 1.016E−154 |
| Signal peptide | 379 | 2824 | 3.996E−134 |
| Postsynaptic neurotoxin | 76 | 99 | 3.356E−89 |
| Phospholipase A2 | 83 | 171 | 1.196E−72 |
| Knottin | 62 | 81 | 3.832E−72 |
| Serine protease inhibitor | 105 | 324 | 1.128E−70 |
| Acetylcholine receptor inhibitor | 60 | 78 | 5.64E−70 |
| Defensin | 77 | 149 | 5.76E−70 |
| Protease inhibitor | 112 | 405 | 3.004E−67 |
| Beta defensin | 50 | 57 | 8.68E−64 |
| Plant defense | 69 | 132 | 6.88E−63 |
| Antimicrobial | 142 | 759 | 3.228E−62 |
| Metal-thiolate cluster | 64 | 123 | 5.52E−58 |
| Antibiotic | 125 | 656 | 3.44E−55 |
| Snake cytotoxin | 38 | 39 | 8.4E−53 |
| Lipid degradation | 71 | 188 | 3.084E−52 |
| Gamma thionin | 39 | 46 | 5.44E−48 |
| Metallothionein superfamily | 41 | 53 | 2.608E−47 |
| S locus-related glycoprotein 1 | 34 | 35 | 7.4E−47 |
| binding pollen coat | | | |
| Whey acidic protein, core region | 44 | 71 | 6.4E−44 |
| Cardiotoxin | 29 | 29 | 3.752E−40 |
| Cyclotide | 27 | 29 | 3.06E−35 |
| Cadmium | 28 | 34 | 3.784E−33 |
| Gamma purothionin | 26 | 29 | 9.32E−33 |
| Vertebrate metallothionein | 29 | 40 | 1.98E−31 |
| Proteinase inhibitor I2, Kunitz metazoa | 33 | 61 | 1.132E−29 |
| Disintegrin | 23 | 25 | 2.136E−29 |
| Cell adhesion | 32 | 62 | 7.8E−28 |
| Calcium | 70 | 409 | 1.28E−26 |
| Cyclotide, bracelet | 19 | 19 | 2.832E−25 |
| Proteinase inhibitor I12, Bowman-Birk | 23 | 33 | 7.16E−24 |
| Fungicide | 45 | 194 | 4.56E−22 |
| Mammalian defensin | 23 | 40 | 5.8E−21 |

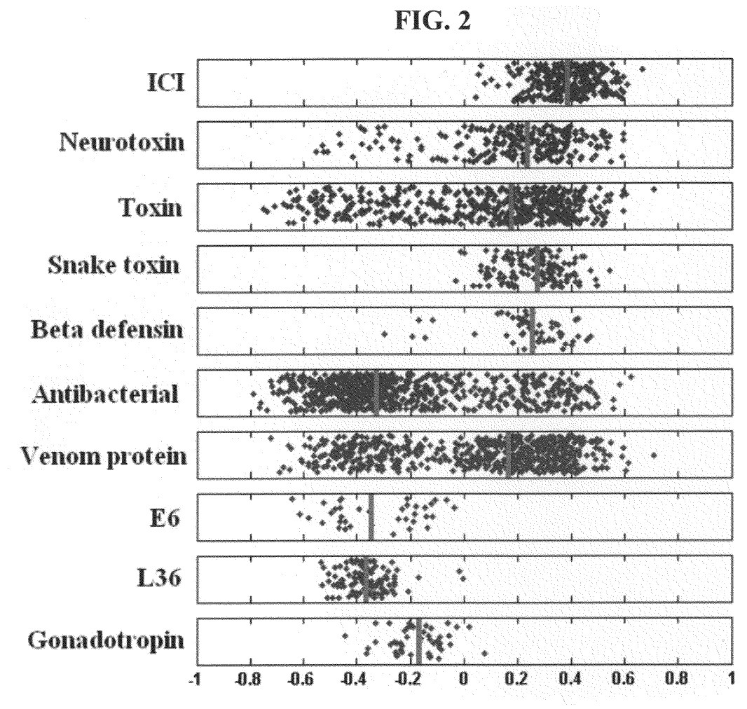

Considering that the training process was performed only on ICIs, it is remarkable to note that several different APT-related functional categories are detected (ICIs, phospholipases, disintegrins, protease inhibitors, etc.). Note that although secreted proteins are enriched, only 13.4% of all secreted proteins are predicted positive, indicating that the classifier does not simply predict all short secreted proteins to be positive. From the score distributions of selected biological groups (FIG. 2), it is apparent that although most toxins obtain positive scores, many do not. This corresponds with the fact that many toxins (as defined by UniProt) belong to the class of structurally stable APTs discussed. Reassuringly, there are specific groups of toxins, such as neurotoxins and snake toxins, which obtain high scores. It was noticed that many false negative predictions occurred in cases where the APT is composed of an extremely long (>60 aa) preprotein with an extremely short (<10 aa) active peptide. In addition to toxins, it is apparent that various antibacterial groups are over-represented. FIG. 2 shows that although antibacterial proteins mostly receive negative prediction scores, certain groups such as β-defensins are generally predicted positive. This corresponds with previous observations on structural and functional similarities between certain classes of antibacterial proteins and APTs [Torres A M, et al., Biochem J 1999, 341 (Pt 3):785-794; Pelegrini P B, Franco O L, Biochem Cell Biol 2005, 37(11):2239-2253]. One over-represented biological group which was suspected initially as false positives is that of the metallothioneins. Metallothioneins are ubiquitous cysteine-rich proteins that have been suggested to possess a variety of functions including zinc homeostasis and antioxidative effects. The full range of functions of these proteins remains unknown. There is no evidence of metallothionein-like toxins, and the high number of cysteines is used in the coordination of heavy metals rather than in the forming of disulfide bonds. However, antibacterial activity of a metallothionein protein expressed in housefly larvae has been reported recently [Jin H Y et al., Acta Biol Hung 2005, 56(3-4):283-295], possibly suggesting that the classification of metallothioneins as incorrect predictions may need to be reconsidered. FIG. 2 shows the prediction results of three groups of short cysteine-rich proteins that do not function as APTs or as APT-like: gonadotropin, L36 ribosomal protein and E6 early regulatory protein families. These groups generally receive negative scores, suggesting that a large amount of cysteines is not sufficient for differentiating between APTs and non-APTs.

In summary, the classifier is apparently able to correctly produce a non-trivial characterization of APT and APT-like proteins. This was confirmed both by cross-validation and evaluation of predictions on a large test set. Reassuringly, it was found that even though the classifier is trained only on ICIs, it was able to detect other groups of non-related APT and APT-like proteins. This finding suggests that this functional super-category, of being APT or APT-like, is not an artificial category that is a union of various smaller functional categories, but rather a genuine biological group that possesses its own unique characteristics. The training of the classifier suggests that a high amount of cysteines is indeed crucial for most proteins of this category, but this feature is evidently not sufficient to define this group. The successful computational characterization of this group enables the detection of novel protein families that are APT or APT-like but do not share sequence or structural similarity with any known proteins.

### Example 2

### Prediction on Honey Bee Proteins

Recently the honey bee genome has been assembled and annotated (Gibbs et al., 2006). The classifier of the present invention was applied to all 10157 protein sequences that were predicted from the honey bee genome.

Materials and Methods

OCLP1 expression assay: RT-PCR was performed on total RNA extracted from head and brain of young honey bees (kindly provided by G. Bloch of the Hebrew University). Oligonucleotide primers were designed to cross an intron/exon to ensure amplification of fully processed RNA. Two pairs were used for the mature OCLP1 (169 nt) and the full length transcript (240 nt).

| | |
|---|---|
| OCLP1 short forward: | |
| 5′TCATGTCCAAGTTTATTCTTC3′ | (SEQ ID NO: 66) |
| OCLP1 short reverse: | |
| 5′AGGAGCTCTTAACACCTGTTCGCA3′ | (SEQ ID NO: 67) |
| OCLP1 long forward: | |
| 5′CTTAATCTTTCCCCTTTCTGC3′ | (SEQ ID NO: 68) |
| OCLP1 long reverse: | |
| 5′AGGAGCTCTTAACACCTGTTCGCA3′ | (SEQ ID NO: 69) |

Results

19 honey bee proteins were predicted to be APT-like proteins by the classifier (Table 5). Of these, 8 are predicted to possess a signal peptide, as expected of APTs. The 4 highest scoring sequences are further described hereinbelow.

TABLE 5

| | Mean | | | | |
|---|---|---|---|---|---|
| Accession | (SD) | SP | Len | InterProScan | Comments |
| GB11222 | 0.46 | − | 29 | — | [Raalin] |
| | (0.11) | | | | |
| GB13285 | 0.44 | + | 50 | — | MCDP (MCDP_APIME) |
| | (0.12) | | | | known bee venom toxin |
| GB19297 | 0.32 | + | 74 | Assasin bug toxin | PHYRE: Omega conotoxin fold |
| | (0.10) | | | | (90%) [OCLP1] |
| GB18161 | 0.32 | + | 46 | — | Apamin (APAM_APIME), |
| | (0.14) | | | | known bee venom ICI |
| GB10910 | 0.27 | − | 48 | EGF-like region | Weak similarity to |
| | (0.08) | | | | metallothionein |
| GB11696 | 0.26 | − | 58 | — | Similarly-lengthed orthologs |
| | (0.14) | | | | found in Drosophila and |
| | | | | | Anopheles |
| GB15018 | 0.23 | + | 76 | Protease inhibitor I8, | Chemotrypsin Inhibitor |
| | (0.13) | | | cysteine-rich trypsin | (AMCI_APIME) |
| | | | | inhibitor-like; EGF-like | |
| | | | | region | |
| GB13221 | 0.23 | − | 79 | Thrombospondin | Probable fragment (gene |
| | (0.09) | | | | prediction error); PHYRE: |
| | | | | | TSP-1 type 1 repeat (95%) |
| GB14748 | 0.22 | − | 47 | Zinc finger, MYND-type | Probable fragment (gene |
| | (0.14) | | | | prediction error); PHYRE: |
| | | | | | Plant lectin/antimicrobial |
| | | | | | peptide (70%) |
| GB15403 | 0.20 | − | 71 | Protease inhibitor I8, | PHYRE: Serine protease |
| | (0.14) | | | cysteine-rich trypsin | inhibitor, ATI-like (95%) |
| | | | | inhibitor-like | |
| GB14111 | 0.19 | + | 56 | — | |
| | (0.11) | | | | |
| GB17579 | 0.19 | + | 90 | Protease inhibitor I8, | PHYRE: Serine protease |
| | (0.12) | | | cysteine-rich trypsin | inhibitor, ATI-like(100%) |
| | | | | inhibitor-like; EGF-like | |
| | | | | region | |
| GB19783 | 0.18 | − | 93 | Protease inhibitor I8, | Api m 6 (ALL6_APIME), |
| | (0.10) | | | cysteine-rich trypsin | known bee venom allergen |
| | | | | inhibitor-like; EGF-like | |
| | | | | region | |
| GB10310 | 0.17 | + | 168 | Whey acidic protein, core | PHYRE: Elafin-like (95%) |
| | (0.11) | | | region | |
| GB13633 | 0.16 | − | 95 | Protease inhibitor I8, | Api m 6 (ALL6_APIME), |
| | (0.12) | | | cysteine-rich trypsin | known bee venom allergen |
| | | | | inhibitor-like; EGF-like | |
| | | | | region | |
| GB14404 | 0.15 | − | 146 | — | PHYRE: Knottin, EGF/laminin |
| | (0.14) | | | | (60%) |
| GB15425 | 0.15 | − | 46 | Zinc finger, PHD type | probable fragment (gene |
| | (0.14) | | | | prediction error); PHYRE: |
| | | | | | PHD zinc finger (100%) |
| GB18697 | 0.14 | − | 144 | — | |
| | (0.13) | | | | |
| GB10134 | 0.13 | + | 74 | Protease inhibitor I8, | PHYRE: Serine protease |
| | (0.09) | | | cysteine-rich trypsin | inhibitor, ATI-like (95%) |
| | | | | inhibitor-like; EGF-like | |
| | | | | region | |

Note
protease inhibitors, WAP proteins, knottin

Apamin and MCDP: Two of the proteins are well-known bee venom toxins, apamin and MCDP, both of which function as K⁺ ICIs [Hughes et al., Proc Natl Acad Sci USA 1982, 79(4):1308-1312; Ziai et al., J Pharm Pharmacol 1990, 42(7):457-461] (note that MCDP performs additional functions). State-of-the-art methods for motif finding and fold recognition, such as InterProScan [Quevillon et al., Nucleic Acids Res 2005, 33(Web Server issue):W116-120] and PHYRE [Kelley et al., J Mol Biol 2000, 299(2):499-520], respectively, failed to detect both of these sequences as toxins. These two predictions suggest that the classifier is able to assign function to proteins beyond the capacity of structure-based or motif-based similarity tools.

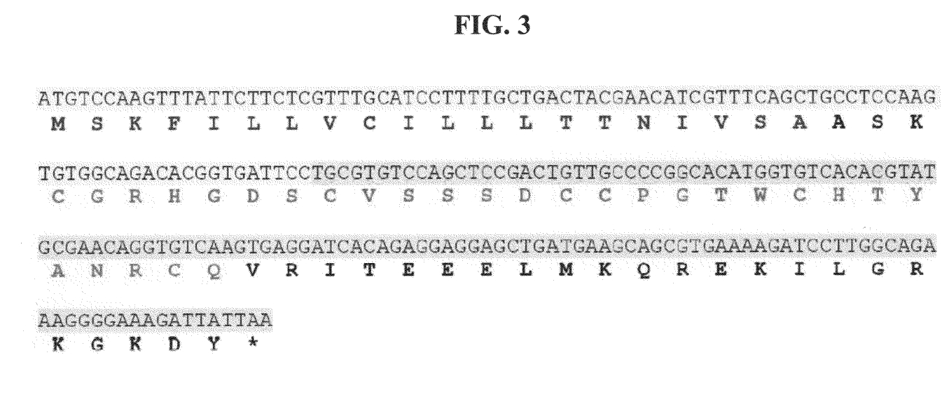

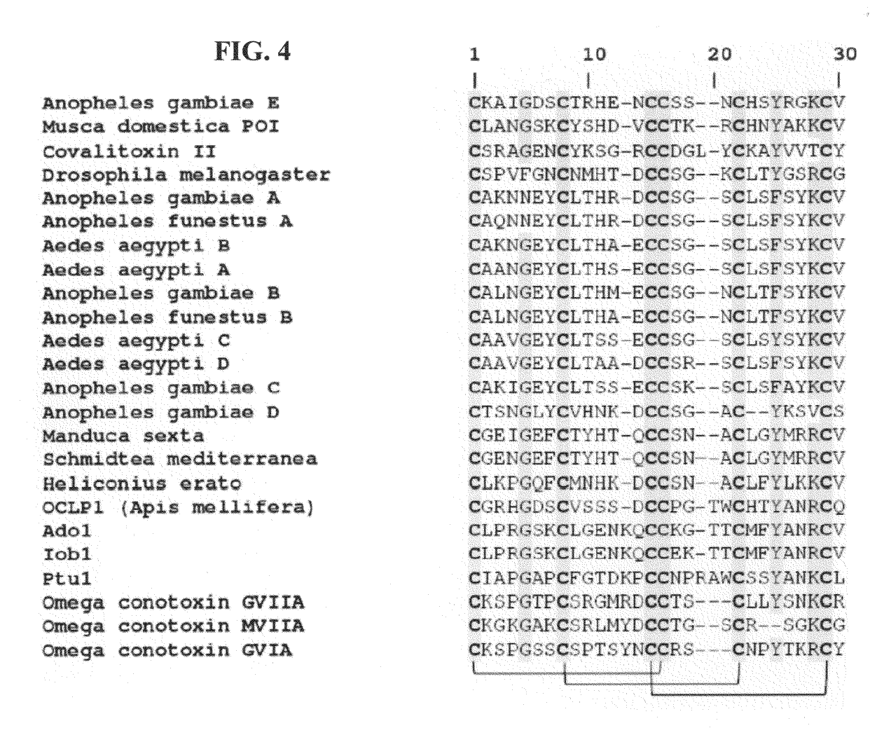

OCLP1 and Raalin: The two remaining protein sequences are putative proteins, referred to herein as OCLP1 (co-conotoxin-like protein 1) and Raalin (after ra'alan, the Hebrew word for toxin), respectively. OCLP1 is a 74 amino acid sequence that possesses a signal peptide followed by a cysteine rich domain of 30 amino acids and an unstructured tail (FIG. 3). An InterProScan search for known sequence motifs indicates that this protein is related to the assassin bug toxins Ptu1, Ado1 and Iob1. These 3 proteins were isolated from the saliva of the assassin bug (Reduviid) species, and were shown to function as voltage-gated Ca²⁺ ICIs and to possess a fold similar to that of ω-conotoxins [Bernard C, et al., Proteins 2004, 54(2):195-205; Bernard C, et al., Biochemistry 2001, 40(43):12795-12800; Corzo G, et al., FEBS Lett 2001, 499(3):256-261]. Multiple sequence alignment of OCLP1 with these assassin bug toxins (FIG. 4) strengthens the notion of homology of these proteins. The multiple sequence alignment shows conservation of the 6 cysteines and of positions G5, T20, Y25, A26, N27 and R28. It has been suggested in the case of the assassin bug toxin that positions K13, Y25 and R28 are functionally important [Bernard C, et al., Proteins 2004, 54(2):195-205; Bernard C, et al., Biochemistry 2001, 40(43):12795-12800]. However, K13 is replaced by an aspartic acid in OCLP1, raising the possibility for interaction with an alternative ion channel as a target.

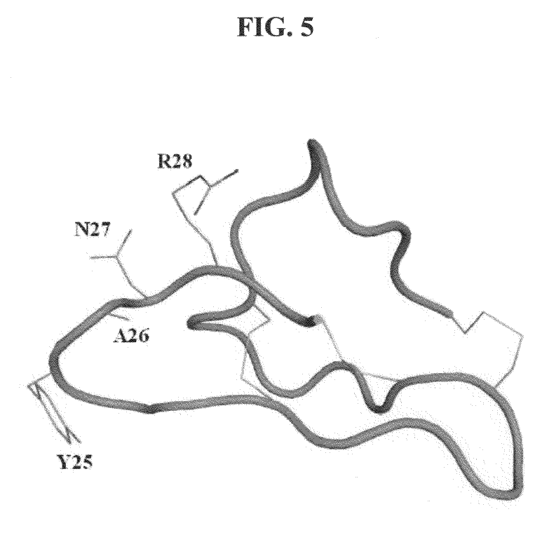

A model of the tertiary structure of OCLP1 was constructed, modeled after the solved structure of Ado1 (PDB 1LMR) (FIG. 5). The side chains of the amino acids in positions 25-28, which are fully conserved in OCLP1 and the three assassin bug ICIs, are exposed at the tip of the protein structure, possibly constituting part of the interface with the ion channel. The PHYRE fold recognition server predicts OCLP1 to possess a fold similar to that of ω-conotoxins and the assassin bug toxin.

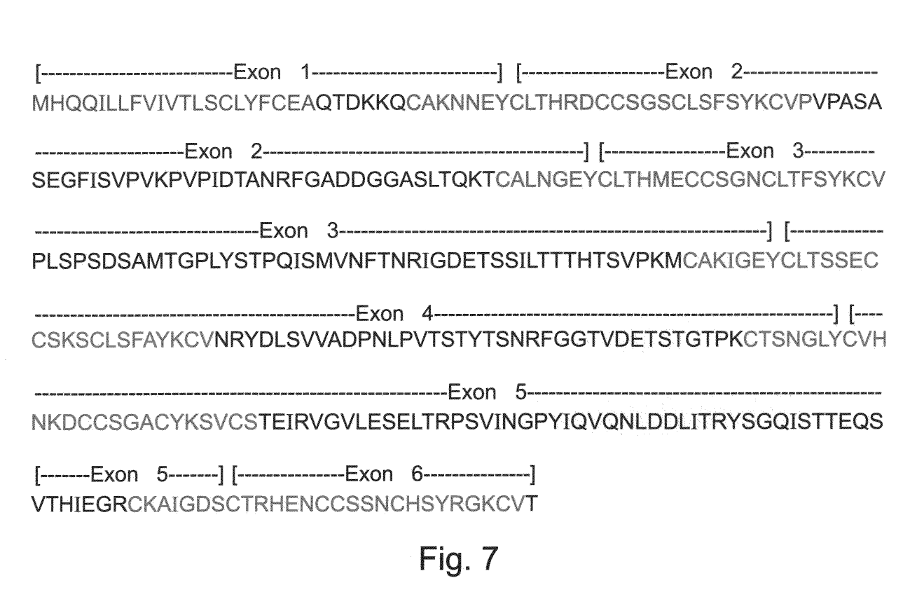

Experimental expression evidence is found for OCLP1 in dbEST [Boguski et al., Nat Genet. 1993, 4(4):332-333]. Remarkably, the EST originates from the bee brain rather than the venom sac, which is located at the bottom of the abdomen. In order to validate expression of OCLP1 in the brain, RT-PCR was performed on RNA extracted from honey bee head and brain. OCLP1 showed a strong expression in the brain (FIG. 6). Searching for additional cDNA evidence, homologs were found in several insects and in S. mediterranea, a flatworm (FIG. 4). The cDNA were obtained from head, whole adult, whole larvae, wing disc and antennae tissues. Of special interest are the A. gambiae and A. aegypti homologs, which both possess signal peptides and are suspiciously long (335 and 372 aa, respectively). Interestingly, both homologs contain multiple repeated occurrences of ω-conotoxin-like (OCL) (5 in A. gambiae and 4 in A. aegypti). Remarkably, in those species for which genomic data is available, it was observed that the locations of the exons were identical relative to the position of the putative OCL peptides, with a splice site located just before the second cysteine of the OCL repeat (compare FIGS. 3 and 7). Multiple sequence alignment of OCLP1, its homologs and various other OCL proteins shows that apart from the 6 conserved cysteine residues, some positions show partial conservation, but only positions G5, Y/F25 and R/K28 are highly conserved (FIG. 4). InterProScan and PHYRE predict all repeats to possess an ω-conotoxin fold.

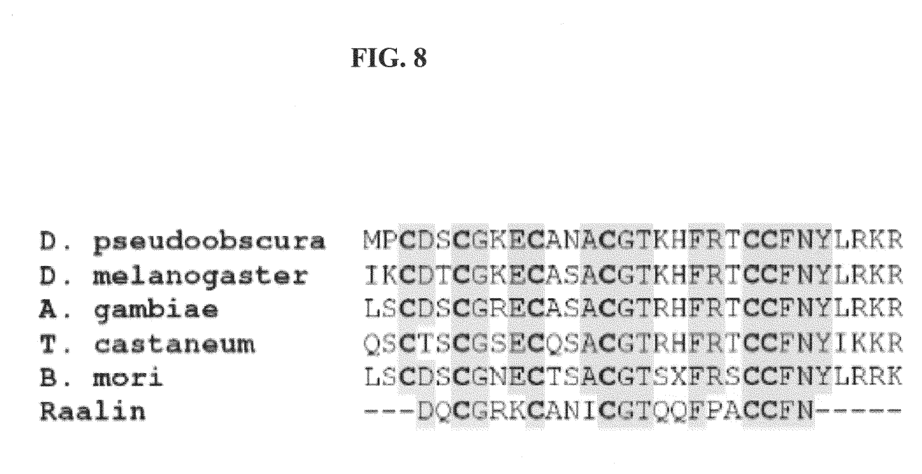

Raalin is a short sequence of 29 aa. Since the predicted ORF does not start with a methionine, it was suspected to be a truncated protein sequence. Several homologs were identified from insect cDNA sequences (FIG. 8). Amongst these is a 108 aa Drosophila melanogaster homolog. Reassuringly, the Drosophila homolog possesses a signal peptide, which is followed by a region of high similarity to Raalin, supporting the notion that the honeybee Raalin sequence is indeed a sequence missing its signal peptide. As for localization of expression, the A. gambiae homolog was found in the head and the B. mori homolog was found in the brain. In all putative homologs, the region of similarity is exclusive to the short cysteine-rich region where the putative peptide is located. No evidence of functional or structural similarity to known APTs was found by structure and sequence prediction tools.

Conclusions

Two putative APT-like bee sequences of hypothetical proteins, OCLP1 and Raalin were discovered. Several evidences provide strong support that OCLP1 is APT-like: It possesses a signal peptide, shares sequence similarity with voltage-gated Ca²⁺ ICIs and is predicted by independent methods to be OCL. Remarkably, this protein is expressed in the brain of the honey bee. Still, some venom toxins are known to be additionally expressed in non-venomous tissues, including the brain [Ma D., Eur Biochem 2001, 268(6):1844-1850]. However, since the bee venom has been studied extensively, it seems unlikely that OCLP1 is a venom toxin. Significant evidence supporting this notion is found in the form of homologs in non-venomous organisms (FIG. 4). In two instances, the homologs contain multiple OCL repeats. This form of multiple repeats of a small peptide is a common form for preproteins of several neuropeptides and of APTs [Kloog Y et al., Science 1988, 242(4876):268-270]. A strong validation for the homology of these proteins is an exact match of exon length and boundaries in these sequences. Although several of the homologs of OCLP1 function as voltage-gated Ca²⁺ ICIs, the Anopheles gambiae and Musca domestica homologs have been previously suggested to function as inhibitiors of melanization by inhibiting phenoloxidase [Daquinag A C, et al., Biochemistry 1999, 38(7):2179-2188; Shi L, et al., Insect Mol Biol 2006, 15(3):313-320].

These functionalities need not necessarily contradict, the biochemical mode of action of these proteins is yet unknown. Multiple sequence alignment suggests that OCLP1 is most similar to the assassin bug ICIs, sharing a unique five amino acid sequence (YANRC) with these proteins (FIGS. 4,5), two of which have been suggested to be critical for ICI function.

Raalin is a 29 amino acid APT-like fragment with homologs in several insects. None of them show any similarity to proteins of known function. Although no known ESTs were found for the bee sequence, in homologs that have data on expression localization, the expression is localized to the brain and head. All full length homologs possess signal peptides. All homologs share a short cysteine-rich region of similarity, while the sequence segments that are not included in the putative mature peptide are not conserved. This is typical for many secreted proteins that undergo post-translational cleavage. It is likely that Raalin does not function as a venom toxin, due to its existence in non-venomous insects and its EST localization to the head and brain.

### Example 3

### Biological Function of OCLP1

Materials and Methods

MS verification: Verification of the band and size determination was preformed by MALDI-TOF as follows: The protein band was excised. The gel plugs were destained with 200 μl of 200 mM ammonium bicarbonate (NH₄HCO₃) pH 8.0 mixed 1:1 with Acetonitrile (AcN) 45 min at 37° C., then the gel pieces were dried completely in SpeedVac. A reduction/alkylation steps were added. The dry gel pieces were rehydrated in 10 μl of 0.02 μg/μl of sequencing grade modified trypsin (Promega) in 10% AcN, 40 mM NH₄HCO₃ pH 8.0 for 1 h at room temperature to allow the trypsin solution to diffuse into the gel pieces. The piece was incubated for 16-18 h at 37° C. Following the digestion, the solution was collected and put in fresh 0.5 ml tube. 50 μl of 0.1% TFA were added to the gel pieces, and sonicated for 15 min. The solution was removed and combined with the solution collected in the previous step. The combined solution was dried completely using SpeedVac and resuspended in 10 μl of 0.1% TFA. This solution was used for MS protein identification. MALDI-TOF MS analysis was performed on a Bruker Daltonics MICROFLEX mass spectrometer (MS). All measurements were performed in positive ion/reflectron mode using standard working protocols. For peptide measurements, α-cyano-4-hydroxycinnamic acid (HCCA) was used as a matrix (Applied Biosystems, CA), utilizing the dried droplet technique. In brief, 0.5 μl of sample solution was mixed with similar volume of the saturated HCCA solution in 30% acetonitrile, 0.1% TFA, spotted on a stainless steel MALDI target and allowed to dry. The mass measurements were performed according to instructions, with trypsin autodigestion peaks used as internal calibrants. The monoisotopic peptide masses were identified using the Bruker TOF Analysis software. The peptide masses were sent to the MASCOT searching software (Matrix Science, London, UK) using the Bruker BioTools software. Each preparation was confirmed by multiple peptide identification before and after cleavage from the cellulaose associated tag.

Bacterial Protein Expression for Large quantities: Escherichia coli B strain BL21(λDE3) was used for overproduction of proteins from plasmids containing T7 promoters and. All plasmids are derivatives of pET22 and pET28a variants (Invitrogen). Plasmids encoding OCLP1 were constructed in this laboratory by PCR amplification from brain bee. Alternatively an oligo-based clone was prepared to ensure changing the codon preference and adopt it for bacterial preferences.

The PCR product was digested with designed restriction enzymes NcoI and Xho1 and cloned into pET22 and pet28a that had been appropriately digested. Standard protocols were used for PCR, restriction digests, ligations, and transformations to TOPO based intermediate plasmid. Plasmid DNA was recovered using a QiaPrep Spin Miniprep kit (Qiagen) following manufacturer's instructions.

All strains were grown in LB medium. When plasmid was present, ampicillin or Kanamycin (ET22 and pET28a, respectively) were added to a concentration of 100 μg/ml. Cultures were induced for protein production at an A₆₀₀ of 0.4 by addition of IPTG to a final concentration ranging from 0.01-1 mM. Growth was allowed to continue for 2-12 hours following addition of IPTG. Uninduced controls were grown in the same way except no IPTG was added. Cells were lysed by boiling in SDS, and proteins were analyzed by SDS polyacrylamide gel electrophoresis. For injection into cells (not exported to the periplasm) a post folding protocol was added. Briefly, after lysis, the fusion protein was solubilized in 6 M guanidinium chloride. The thiols were protected by forming mixed disulfides with glutathione and the fusion protein was cleaved with the appropriate protease. The peptide was treated with dithiothreitol to reduce the mixed disulfides. After these treatments, the reduced protein was allowed to fold and form disulfides for 24 hr in the presence of 1 mM GSSG and 2 mM GSH at pH 7.3, 25° C. The folded protein identity was confirmed by mass spectrometry and the functional Ca²⁺ channel-binding assay.

For high expression of the protein, BL21(λDE3) was transformed. Colonies were picked directly from the transformation plate and inoculated into 5 ml LB containing ampicillin for overnight growth. The overnight culture was diluted 1:100 into fresh LB with ampicillin and grown to an A₆₀₀ of 0.4-0.5 for induction. For continuous subculturing experiments, samples were removed before addition of IPTG and used to inoculate fresh LB plus ampicillin media at a dilution of 1:200.

Xenopus oocytes injection and recordings: Stage V and VI oocytes were surgically removed from anesthetized adult Xenopus laevis and treated for 2-3 hr with 2 mg/ml collagenase (Type IA, Sigma) in a Ca-free medium. After a recovery period of 10 hr, nuclear injection was performed using 10 nI of a 1:1:1 mix of cDNAs encoding rat brain Ca channel α₁, α₂, and β subunits inserted into the Xenopus expression vector. Before recording, oocytes were incubated at 19° C. under gentle shaking on a rotating platform for 4 days in standard saline (in mM): 100 NaCl, 2 KCl, 1.8 CaCl₂, 1 MgCl₂, 5 HEPES, at pH 7.5 containing 2.5 mM sodium pyruvate and 10 μg/ml gentamycin sulfate.

For oocytes, macroscopic currents were recorded using the two-electrode voltage-clamp technique with either Axoclamp amplifier (Axon Instruments, USA). Acquisition and data analysis were performed using Axon Instruments software. Leak currents and transients were subtracted. Oocytes were placed in a 150 μl recording chamber and superfused continuously with a solution containing (in mM): either 5 Ba(OH)₂, 5 Ca(OH)₂, or 5 Sr(OH)₂, 60 TEA-OH, 25 NaOH, 2 CsOH, 5 HEPES (titrated to pH 7.3 with methane sulfonic acid). Pipettes of typical resistance ranging from 0.5 to 1.5 MΩ were filled with 2.8 M CsCl, 0.2 M CsOH, 10 mM HEPES, and 10 mM BAPTA-free acid. For each oocyte, solutions were switched from Ba to Ca to Sr and then again to Ba to eliminate possible errors arising from rundown during the time course of the experiment.

The experiments were carried out according to protocols established by Alomone laboratory (Jerusalem).

Each experiment was conducted with 8 independent injected oocytes for the experiments and another 8 for controls. The effect of OCLP1 was applied with addition of 1:200 of the product following in vitro folding protocol of column eluted product following cleavage of the Cellulose based tag.

Injection into fish muscle: Fish (Gumbusia affinis) were obtained from freshwater ponds. For fish assays, 5 ml aliquots were injected below the dorsal fin in the rear part of Gambusia of 250 mg body mass. The paralytic dose (PD50) was determined 30 min following injection. Paralysis was defined as any locomotory disturbance which prevents the animal from moving and changing its location. Fish were observed for up to 24 h following injection.

Injection into insects: Laboratory bred blowfly larvae (Sarcophaga falculata) were used for insect bioassays. 5 ml aliquots were injected and the behavior of the larvae was analyzed.

Results

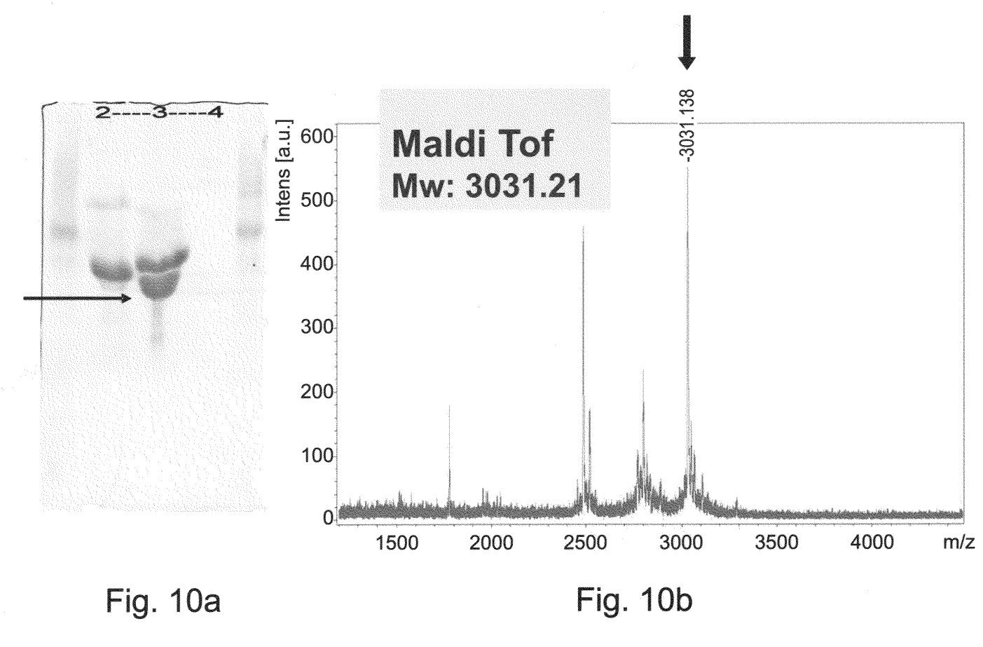

Bacterial protein expression: FIG. 10 illustrates the expression of OCLP1 in bacteria.

Injection into insects: No detected activity in ‘behavior’—larvae are vital also 24 hrs later.

OCLP1 injection into Xenopus oocytes: As illustrated in FIGS. 11A-D, a change of ˜10% in current flow is consistently recorded for the OCLP1 and not for the buffer only and oxidized OCLP1.

OCLP1 injection into fish: Short and long term effects were recorded. Positive control experiment was done by purified toxin (extracted from a paralytic and cytolytic protein from a Hydra, provided by Prof. Zlotkin). The positive control reached lethality of the fish after 4 hrs. OCLP1 was injected for 7 fish and 5 injected as controls. Paralysis phenotype was evident for 5 fish and none was affected in the controls. A full recovery after 6-8 hours was monitored for 6 fish (additional fish died following jumping from the water). All negative controls recovered with no obvious phenotype once injection with the oxidized OCLP1 (2 fish) and injection of the buffer alone (3 fish).

### Example 4

### Prediction on Mouse Proteins

FANTOM is a newly available resource for the mouse transcriptome, with thousands of previously unreported transcripts [Carninci et al., Science 2005, 309(5740):1559-1563. Amongst these are 5154 sequences that have been identified as novel proteins. The classifier of the present invention was applied to all 5154 protein sequences.

Results

16 of the 5154 novel FANTOM sequences were predicted by the classifier of the present invention to be APT-like. Table 6 below summarizes the 16 predicted sequences.

TABLE 6

| | Mean | | | | |
|---|---|---|---|---|---|
| Accession | (SD) | SP | Len | InterProScan | Comments |
| Q3V2E2_MOUSE | 0.52 (0.19) | − | 62 | Vertebrate | Metallothionein 4 |
| | | | | metallothionein | (MT4_MOUSE) |
| Q3UKY8_MOUSE | 0.37 (0.19) | + | 63 | EGF-like region | |
| Q3UQE2_MOUSE | 0.33 (0.15) | + | 69 | Beta defensin | Beta defensin 1 |
| | | | | | (BD01_MOUSE) |
| Q3UW41_MOUSE | 0.33 (0.06) | + | 83 | — | WFDC9; PHYRE: |
| | | | | | Scorpion toxin-like |
| | | | | | fold (45%) |
| Q3UW09_MOUSE | 0.30 (0.07) | + | 88 | Proteinase inhibitor | |
| | | | | I2, Kunitz metazoa | |
| Q3V490_MOUSE | 0.29 (0.09) | + | 66 | — | Beta defensin 27 |
| Q3U2W8_MOUSE | 0.28 (0.11) | + | 75 | Phospholipase A2, | |
| | | | | active site | |
| Q3V491_MOUSE | 0.28 (0.14) | + | 67 | — | Beta defensin 36 |
| Q3UG05_MOUSE | 0.27 (0.15) | + | 142 | Phospholipase A2 | Group IIE secretory |
| | | | | | phospholipase A2 |
| | | | | | (PA2GE_MOUSE) |
| Q4QY32_MOUSE | 0.25 (0.10) | + | 63 | — | Beta defensin 51 |
| Q3USP9_MOUSE | 0.20 (0.17) | − | 68 | Vertebrate | Metallothionein 3 |
| | | | | metallothionein; | (MT3_MOUSE) |
| | | | | Whey acidic protein, | |
| | | | | core region | |
| Q4KXB6_MOUSE | 0.19 (0.16) | + | 126 | Whey acidic protein, | WAP1/WFDC5; |
| | | | | core region; | PHYRE: Elafin-like |
| | | | | Proteinase inhibitor | (100%) |
| | | | | I2 | |
| Q3UF02_MOUSE | 0.14 (0.13) | + | 53 | — | |
| Q3UW31_MOUSE | 0.11 (0.11) | + | 111 | Snake toxin-like | PHYRE: Snake toxin- |
| | | | | | like fold (85%) |
| | | | | | [ANLP1] |
| Q3TNQ5_MOUSE | 0.10 (0.08) | + | 70 | — | |
| Q3T9Y6_MOUSE | 0.10 (0.10) | + | 54 | — | |

Of these, 14 possess a signal peptide. One of these sequences, is a 111 aa sequence which is referred to herein as mANLP1 (mouse α-neurotoxin like protein 1). mANLP1 possesses a signal peptide and is identified by both InterProScan and PHYRE as ‘snake toxin-like’ (also known as the 3 finger toxin fold). By searching the physical neighbourhood of the MANLP1 gene, other genes were also identified as encoding toxin like proteins. Table 7 summarizes the mouse sequences clustered in chromosome 9 (in a <1 million bases) and the human homologs thereof clustered in Chromosome 11.

TABLE 7

| | GeneBank/ | | | | UniProt | |
|---|---|---|---|---|---|---|
| | symbol | Alternative | | Signal | Accession | Expression |
| Name^{a} | (# of sequences) | transcript | Location | Peptide | (aa) | evidence |
| mANLP1 | Gm846 | AK144787 | chr9: 35,319,955 | S | Q3UW31 | epididymis, |
| | | | | | | lung |
| Seminal | 9530004K16Rik | AF134204 | chr9: 35,357,257 | S | Q9R018 | epididymis, |
| vesicle | caltrin, SVS7 | | | | SVS7_MOUSE | brain |
| protein 7 | | | | | | |
| mANLP2 | D730048I06Rik | AK033813 | chr9: 35,537,721 | S | Q9CQB8 | mammary |
| | | | | N | Q3UW50 | gland, |
| | | | | | | epididymis |
| mANLP3 | 9230110F15Rik | AK020329 | chr9: 35,588,000 | S | Q9D262 | epididymis |
| mANLP4 | AK136639 | AK033758 | chr9: 35,597,526 | S | Q8CC74 | epididymis |
| mANLP5 | ENSF00000014716 | AK020345 | chr9: 35,658,094 | S | No report | epidydymis |
| | | | | N | No report | |
| mANLP6 | ENSF00000014716 | | chr9: 36,006,890 | S | No report | |
| mANLP7 | ENSMUSP00000048154 | pseudogene | chr9: 36,136,495 | N | | predicted |
| mANLP8 | LOC434396 | AK136744 | chr9: 36,282,074 | S | Q3UW02 | epididymis |
| | | | | | | sperm, |
| | | | | | | testis |
| Secreted | Gm191, SSLP1, | AK144443 | chr9: 36,385,426 | S | Q3UN54 | seminal |
| seminal- | A630095E13Rik | | | | | vesicles. |
| vesicle | | | | | | |
| Ly-6 | | | | | | |
| protein 1 | | | | | | |
| Acrosomal | ACRV1, Msa63 | AK030129 | chr9: 36,442,921 | S | ASPX_MOUSE | spermatid, |
| protein | | | | | (261) | testis |
| SP-10 | | | | | Q9DAM6 | epididymis |
| (precursor) | | | | | P50289 | |
| acrosomal | ACRV1 | 11 alt. | chr11: 125,051,796 | S | P26436 | acrosome, |
| vesicle | | splicing | | | | testis, |
| protein 1 | | | | | | muscle, |
| isoform | | | | | | |
| (precursor) | | | | | | |
| hANLP1 | PATE | AF462605 | chr11: 125,121,398 | S | Q8WXA2 | prostate, |
| | | | | | | testis, brain |
| hANLP2 | LVLF3112 | C11orf38 | chr11: 125,152,446 | S | Q6UY27 | secretion |
| hANLP3 | AK123042 | FLJ41047 | chr11: 125,208,421 | S | | prostate |

The gene cluster consists of several gene products that are related to the Ly6-uPAR family. All genes in the cluster posses a signal peptide but lack a GPI anchor that is characteristic for other members of the Ly6-uPAR family. Current expression evidence shows that ANLP genes are mostly expressed in the testis. Some gene transcripts were also detected in lung and brain tissue.

### Example 5

Biological Activity of Mouse ANLP1

Materials and Methods

P19 cells were originally from M. W. McBurney (University of Ottawa, Canada, 1983). Cells were cultured and differentiated essentially as described (Parnas and Linial, 1995, Int J Dev Neurosci. 1995 November; 13(7):767-81). Briefly, cells were aggregated in the presence of 0.5 μM RA (Sigma) for 4 days. At day 4, the aggregates were treated with trypsin (0.025%, 5 min, 37° C.) and plated on culture-grade plates coated with poly-lysine (10 μg/ml, Sigma). The cells were plated in defined medium—DMEM supplemented with BioGro2 (25 μg/ml transferrin, 1 μg/ml insulin, 15 nM selenium, 20 mM ethanolamine, 10 mM Hepes, pH 7.3) supplemented with 1 μg/ml fibronectin. Cytosine-β-D-arabinofuranoside (Ara-C, 5 μg/ml, Sigma) was added 1 day after plating, for 2 days. Medium (without fibronectin) was replaced every 48 h. All media and sera products were purchased from Biological Industries Co. (Israel). All media were supplemented with 3.5 mM glutamine and with antibiotics (Penicillin, Streptomycin and Amphotericin B). After 2 more days P19 aggregates (4 days old) cells were trypsinized and plated (0.5-1×10⁶ cells) and AraC (5 μg/ml) was added 24 h later.

Results

As illustrated in FIGS. 12A-D ANLP-1 is up-regulated during neuronal differentiation by retinoic acid.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated polynucleotide comprising a nucleic acid sequence encoding a polypeptide which comprises an amino acid sequence selected from the group consisting of SEQ ID NOs: 1, 3-12, 31-35, 39-46, 57-59.

2. An isolated polynucleotide comprising a nucleic acid sequence encoding a polypeptide which comprises an amino acid sequence at least 90% identical to a sequence as set forth in selected from the group consisting of SEQ ID NOs: 1, 3-12, 31-35, 39-46, 57-59 wherein said polypeptide comprises an ion channel modulatory activity.

3. The isolated polynucleotide of claim 1, wherein said polypeptide comprising an amino acid sequence as set forth in SEQ ID NO: 2.

4. (canceled)

5. The isolated polynucleotide of claim 1, wherein said nucleic acid comprises a sequence selected from the group consisting of SEQ ID NO: 13, 14, 36-38, 39-46 and 57-59.

6. The isolated polynucleotide of claim 1, wherein said nucleic acid sequence is selected from the group consisting of SEQ ID NOs: 15-17.

7. An isolated polypeptide comprising an amino acid sequence selected from the group consisting of SEQ ID NO: 1, 31-35, 39-46 and 57-59 and an amino acid sequence at least 90% identical to a sequence as set forth in 1, 31-35, 39-46 and 57-59, wherein said polypeptide comprises an ion channel modulatory activity.

8. The isolated polypeptide of claim 7, comprising an amino acid sequence as set forth in SEQ ID NO: 2.

9. An isolated polypeptide comprising an amino acid sequence selected from a group consisting of SEQ ID NOs: 3-12.

10-15. (canceled)

16. A pharmaceutical composition comprising a pharmaceutically acceptable carrier and as an active ingredient an isolated polypeptide, which comprises an amino acid sequence having a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue.

17. A pesticidal composition comprising an agriculturally acceptable carrier and as an active ingredient an isolated polypeptide, wherein an amino acid sequence of said isolated polypeptide confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue.

18-20. (canceled)

21. A method of treating a nerve disease or disorder, the method comprising administering to a subject in need thereof a therapeutically effective amount of a polypeptide comprising an amino acid sequence, wherein said amino acid sequence confers to a consensus sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀ wherein X₁, X₈, X₁₅, X₁₆, X₂₂ and X₂₉ comprise a cysteine residue, thereby treating the nerve disease or disorder.

22-26. (canceled)

27. The pharmaceutical composition of any of claim 16, wherein X₂ is a hydrophobic amino acid, X₅ is a small amino acid, X₆ is a turnlike amino acid, X₉ is a hydrophobic amino acid, X₁₁ is a polar amino acid, X₁₂ is a turnlike amino acid, X₁₄ is a polar amino acid, X₁₇ is a small amino acid, X₂₀ is a turnlike amino acid, X₂₃ is a hydrophobic amino acid, X₂₅ is an aromatic amino acid, X₂₈ is a positive amino acid and X₃₀ is a hydrophobic amino acid.

28. The pharmaceutical composition of claim 16 wherein X₂ is a hydrophobic amino acid, X₅ is glycine, X₆ is a polar amino acid, X₉ is a hydrophobic amino acid, X₁₀ is a turnlike amino acid, X₁₁ is a polar amino acid, X₁₂ is a turnlike amino acid, X₁₄ is a polar amino acid, X₁₇ is a small amino acid, X₂₀ is a turnlike amino acid, X₂₁ is a turnlike amino acid, X₂₃ is a hydrophobic amino acid, X₂₄ is a small amino acid, X₂₅ is an aromatic amino acid, X₂₆ is a turnlike amino acid, X₂₈ is a positive amino acid and X₃₀ is an aliphatic amino acid.

29. The pharmaceutical composition claim 16, wherein X₂ is a small amino acid, X₃ is a turn-like amino acid, X₄ is a small amino acid, X₅ is glycine, X₆ is a polar amino acid, X₇ is a hydrophobic amino acid, X₉ is a hydrophobic amino acid, X₁₀ is a small amino acid, X₁₁ is a polar amino acid, X₁₂ is a small amino acid, X₁₄ is a polar amino acid, X₁₇ is serine, X₂₀ is a small amino acid, X₂₁ is a small amino acid, X₂₃ is a hydrophobic amino acid, X₂₄ is a small amino acid, X₂₅ is an aromatic amino acid, X₂₆ is a tiny amino acid, X₂₇ is a hydrophobic amino acid, X₂₈ is a positive amino acid and X₃₀ is valine.

30. The pharmaceutical composition of claim 16, wherein X₂ is a tiny amino acid, X₃ is a turn-like amino acid, X₄ is a small amino acid, X₅ is glycine, X₆ is a negative amino acid, X₇ is an aromatic amino acid, X₉ is an aliphatic amino acid, X₁₀ is a small amino acid, X₁₁ is a charged amino acid, X₁₂ is a small amino acid, X₁₄ is a negative amino acid, X₁₇ is serine, X₂₀ is a small amino acid, X₂₁ is a small amino acid, X₂₃ is leucine, X₂₄ is an alcoholic amino acid, X₂₅ is tyrosine, X₂₆ is a tiny amino acid, X₂₇ is a hydrophobic amino acid, X₂₈ is lysine and X₃₀ is valine.

31. The pharmaceutical composition of claim 16, wherein X₂ is a tiny amino acid, X₃ is a turn-like amino acid, X₄ is a small amino acid, X₅ is glycine, X₆ is Glutamic acid, X₇ is an aromatic amino acid, X₉ is lysine, X₁₀ is an alcoholic amino acid, X₁₁ is histidine, X₁₂ is a small amino acid, X₁₄ is a negative amino acid, X₁₇ is serine, X₂₀ is a small amino acid, X₂₁ is a tiny amino acid, X₂₃ is leucine, X₂₄ is an alcoholic amino acid, X₂₅ is tyrosine, X₂₆ is a tiny amino acid, X₂₇ is a turn-like amino acid, X₂₈ is lysine and X₃₀ is valine.

32. The pharmaceutical composition of claim 16, wherein X₅ is glycine, X₆ is a turnlike amino acid, X₉ is a hydrophobic amino acid, X₁₀ is a turnlike amino acid, X₁₁ is a polar amino acid, X₁₂ is a turnlike amino acid, X₁₇ is a polar amino acid, X₂₅ is an aromatic amino acid, X₂₆ is an turnlike amino acid and X₂₈ is a polar amino acid.

33. The pharmaceutical composition of claim 16, wherein X₂ is a hydrophobic amino acid, X₅ is glycine, X₆ is a turnlike amino acid, X₉ is a hydrophobic amino acid, X₁₀ is a turnlike amino acid, X₁₁ is a polar amino acid, X₁₂ is a small amino acid, X₁₄ is a polar amino acid, X₁₇ is a small amino acid, X₂₀ is a turnlike amino acid, X₂₃ is a hydrophobic amino acid, X₂₅ is tyrosine, X₂₆ is a small amino acid, X₂₈ is a positive amino acid and X₃₀ is a hydrophobic amino acid.

34. The pharmaceutical composition of claim 16, wherein X₂ is a hydrophobic amino acid, X₃ is a small amino acid, X₄ is a hydrophobic amino acid, X₅ is glycine, X₆ is a polar amino acid, X₉ is a hydrophobic amino acid, X₁₀ is a turnlike amino acid, X₁₁ is a polar amino acid, X₁₂ is a small amino acid, X₁₄ is a polar amino acid, X₁₇ is a small amino acid, X₂₀ is a turnlike amino acid, X₂₁ is a turnlike amino acid, X₂₃ is a hydrophobic amino acid, X₂₄ is a small amino acid, X₂₅ is tyrosine, X₂₆ is a tiny amino acid, X₂₈ is a positive amino acid and X₃₀ is a small amino acid.

35. The pharmaceutical composition of claim 16, wherein X₂ is a turnlike amino acid, X₃ is a small amino acid, X₄ is a hydrophobic amino acid, X₅ is glycine, X₆ is a polar amino acid, X₉ is a hydrophobic amino acid, X₁₀ is a small amino acid, X₁₁ is a polar amino acid, X₁₂ is a small amino acid, X₁₄ is a polar amino acid, X₁₇ is a small amino acid, X₂₀ is a turnlike amino acid, X₂₁ is a polar amino acid, X₂₃ is a hydrophobic amino acid, X₂₄ is a small amino acid, X₂₅ is tyrosine, X₂₆ is a tiny amino acid, X₂₇ is a small amino acid, X₂₈ is a positive amino acid and X₃₀ is an aliphatic amino acid.

36. The pharmaceutical composition, of claim 16, wherein X₂ is a tiny amino acid, X₃ is a tiny amino acid, X₄ is a small amino acid, X₅ is glycine, X₆ is a negative amino acid, X₇ is a polar amino acid, X₉ is an aliphatic amino acid, X₁₀ is a small amino acid, X₁₁ is a small amino acid, X₁₂ is a small amino acid, X₁₄ is a negative amino acid, X₁₇ is serine, X₂₀ is a small amino acid, X₂₁ is a small amino acid, X₂₃ is a hydrophobic amino acid, X₂₄ is an alcoholic amino acid, X₂₅ is tyrosine, X₂₆ is alanine, X₂₇ is a small amino acid, X₂₈ is lysine and X₃₀ is valine.

37. The pharmaceutical composition, of claim 16, wherein said isolated polypeptide comprises an amino acid sequence selected from the group consisting of SEQ ID NOs: 1-12 and SEQ ID NOs: 20-30.

38-57. (canceled)


SEQUENCE LISTING (69 sequences)

SEQ ID NO: 1 - protein, 28 aa, Apis mellifera
CGRHGDSCVSSSDCCPGTWCHTYANRCQ

SEQ ID NO: 2 - protein, 74 aa, Apis mellifera
MSKFILLVCILLLTTNIVSAASKCGRHGDSCVSSSDCCPGTWCHTYANRCQVRITEEELMKQREKILGRKGKDY

SEQ ID NO: 3 - protein, 27 aa, Aedes aegypti
CAANGEYCLTHSECCSGSCLSFSYKCV

SEQ ID NO: 4 - protein, 27 aa, Aedes aegypti
CAKNGEYCLTHAECCSGSCLSFSYKCV

SEQ ID NO: 5 - protein, 27 aa, Anopheles funestus
CALNGEYCLTHAECCSGNCLTFSYKCV

SEQ ID NO: 6 - protein, 27 aa, Aedes aegypti
CAAVGEYCLTSSECCSGSCLSYSYKCV

SEQ ID NO: 7 - protein, 27 aa, Musca domestica
CLANGSKCYSHDVCCTKRCHNYAKKCV

SEQ ID NO: 8 - protein, 27 aa, Heliconius erato
CLKPGQFCMNHKDCCSNACLFYLKKCV

SEQ ID NO: 9 - protein, 27 aa, Manduca sexta
CGEIGEFCTYHTQCCSNACLGYMRRCV

SEQ ID NO: 10 - protein, 27 aa, Schmidtea mediterranea
CGENGEFCTYHTQCCSNACLGYMRRCV

SEQ ID NO: 11 - protein, 27 aa, Aedes aegypti
CAAVGEYCLTAADCCSRSCLSFSYKCV

SEQ ID NO: 12 - protein, 27 aa, Anopheles funestus
CAQNNEYCLTHRDCCSGSCLSFSYKCV

SEQ ID NO: 13 - DNA, 84 bp, Apis mellifera
tgtggcagacacggtgattcctgcgtgtccagctccgactgttgccccggcacatggtgtcacacgtatgcgaacaggtgtcaa

SEQ ID NO: 14 - DNA, 224 bp, Apis mellifera
atgtccaagtttattcttctcgtttgcatccttttgctgactacgaacatcgtttcagctgcctccaagtgtggcagacacggtgattcctgcgtgtccagctccgactgttgccccggcacatggtgtcacacgtatgcgaacaggtgtcaagtgaggatcacagaggaggagctgatgaagcagcgtgaaaagatccttggcagaaaggggaaagattatta

SEQ ID NO: 15 - DNA, 1718 bp, Aedes aegypti
ggtcgtcggaagcgcaactcggaactctaagcgatccagtgatcaatacagtgctcgaaggaaatcgattgaatttcaacagtggatagtgtgtttgttttggagaaacctgttgcaccagattagcaaaaaaaaaacaagtcgaactaagggcagtgttgaaggaaaggtcgaaagttgttttgcagttttcggaaatgaagcagttgatcttgctgctcgtagtggcagtggcgctggtcgattacagccaggctcagggcaatcgaaagtgcgcggcgaacggagaatattgtttgacccattccgaatgctgctcgggcagctgtttgagtttttcctacaaatgcgtccctgtgccaccgagtgccagcgttggaaccgtattcgttccagcccccattgagacagataatcggttcggaggcggagacgacagcggaaccagcatcacacaaaaaacctgtgccaaaaatggcgaatattgcctgaccgcagccgattgctgttcccggagttgcctgagcttttcgtacaagtgcgtccagaactacgacctgggcacccaacagttgacgtcatcgggaattccagtgcagctgcctgtcggcggatcgtccatcaacaccatcgacacggctaatcgcttcggaggtggtggctccgaaagacaatgtctcgctaatggacacgcgtgtttctacggccaggagtgctgctctggggcctgcttcagatcgttctgcgccacccaaatccacctgggaatccccgaatcggcactgactcgaccgtccgccgtaaatggcccgttcgtgcaggtcaacagcttggacgagttgataactcgctttggtggaggcagtgatgccaaccatgcccgggattccagcgccagtgcttcgttgaagcgggccaatatcggggcgaggagcggcgttgagaagcagtgtgccgtcgttggcgaagggtgctcccgacaggaagattgctgctccatgagatgtcattcgtacaggcgcaagtgtgtcacgtagagagaggagttcgaacgcaccaacaagcctcacacaatagagagagagaggtgctcgtgcggatgaggagctgatcagtggatgctgggagcagaaaaaaaaactcccacgaaaagacattgtaatgttttattttatcgatggaggaatgtggtaaaaacggcggattagacacagaagcacccaaagaaaaaagttaatgcagttcggcacccggggtgcgtacaattgcgtttaaggtaaaggtagaacgaagccagaatccggactttgaagagcacaaatcaagagaattctagattatggcttcttatcttcttcacctttagcgcaaattgtaagtgccccggcccaaatccgaattgcatccaaatctgacagcctccggttgggtgctccggaccgggatattttgagccaatcgatgcaatcggcatcattcggattgttaataatttttcatggaacagccgccatctgccaccttccttcctccagtttgcttttattttatattttattcccttcaatcaacattttttattaaacatatattttgctgtgatgaaacacgatcctagagcagaagagtggatgtgtttggagagtatgagtgagtgagcaagagcgagcgagacggcgagaatgtgatttagtggaaataaatcataaatgaaacag

SEQ ID NO: 16 - DNA, 1157 bp, Anopheles funestus
gaaatagcaagatcacttaaattatactaggtagatagctgttttggacattgttactaacacacatttcacgaaagcgaagttgtagcaagttaattgaagataaaatatgcatcagcagatattgttgtttgttatagtgacactgagttgtttatatttctgtgaagcgcaaacagataaaaaacaatgtgcaaaaaataatgaatattgtcttactcatcgagattgttgttcaggaagttgcttaagcttctcatataaatgcgtacctgtgccagcaagtgcttctgaaggatttataagcgttccagtgaagccagttccaattgatacagcaaatcgtttcggagcagatgatggtggtgcaagtttaacccaaaagacatgcgctctgaacggagaatattgtttgacgcatatggaatgttgttcaggcaattgtttaactttctcttataaatgtgtgcctctcagtccatctgattctgccatgactgggccactctattcaacaccccagatctcaatggtaaactttacgaatcgaataggcgatgaaacttcatctattttaacaacaacacatacttcagttccaaaaatgtgtgctaaaattggtgaatattgtttaacatcttcggaatgctgctccaaaagttgtttaagctttgcgtataaatgcgtcaacagatatgacctttcagtagtggcagatccaaatctacctgtaacatcaacttatacttccaatcgttttgggggtacagtagacgaaacaagcacaggaacacccaagtgcacatcgaacggattatattgtgtccacaacaaagattgctgttccggagcatgttataaatctgtatgttcaacagagatccgtgttggtgtgctggaatcagagttaactcgtccgtcggttataaatggaccatatattcaagtacaaaatttagatgacctcattacccgttacagtggacaaatttcaacgacagagcaaagcgttactcatatagaagggcggtgtaaagctattggtgatagctgtacccggcatgaaaattgctgttcttcaaactgtcattcatacaggggaaagtgtgttacctaaattaaacttcataacaatttcgaaaaaataaaaatttact

SEQ ID NO: 17 - DNA, 612 bp, Manduca sexta
acagaatccgttagtggtaaactgtattcgaccacaatgtctcgtgtttgcatgttgttcttttgcatcgtgtttgtatacgcgagtggcaatttgataagggacggagttgatgacccgtcagtgacaacaaaggaaattgttgtgcctaaagatgtcgaagatctagacccgattcctgttgtggaaccgcagattgaaactacgacgaagaaatgtggtgaaattggcgaattttgcacgtaccacacacaatgctgcagcaacgcttgcctcggctacatgcggagatgcgtgtctggctccggatagaaaacacttatttcttattactttttagttaaaaaaaaaacattatttattaattttacgaattggatgtatcggatggtccatgtgtgtcaagtcaggcgggttcgcggcgaactccatgatctctttcatagcaccttcggaaattctccatagttctggatagtcccttttgaaatcattgtaccatttgtgtatctggaaaagaaataaaaatatatttttaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 1603 bp, Drosophila melanogaster
ctagctggcaaagagtcggtgactaaagtcaacaactggcggactctgccaaacactctgcactgaatcggcagccaccatgtttcgatagtcggctactccgcgaaacaggctgagaaacatgttggtgaacatggtaacctcatcctggttgagaggattaagctgctgctgcagtggtttaaactccacaattattgcccatccgacctagacgcaattatcaattggaattgttggacatgctcctctgtttgcccaagtcaagaagtcgggttacatttgggtcaagacgtccgagataattgggaattgccaatgtcaatatttaatgtcaacgcatcagttacgatacacatatatatagatatagaacgagatatgggtagaataacgtgacctatgttgaataatcagcgtttaagtaccgttgaggtgtgccctctgaattatttgttctgagcttaagtaggttaaggttactttaagtgtttttcatatctgtaaattttctgttctaattgttaaagaaatatttcttcctttgaatagataaacagaaaagaaactgaatagttttcctgccaataaaaagcataactattctgcctaatctgtacatatattctatagtgtatctttttattataaaaaccaagatttcccatgatatgaacagccttggccacaacagccacaacttaaccttaactaaccttaattattatggaaataaaaccatttatcagttcagtactgggaatttttgccatcttcaaatgtcccgcaatgtagctatctattgtaaatcgcttactgacagttcaagattaagcaaaacaatgagtatgtatggtttatttatttattgtgatattgtgacaaaactatatacaagttttcagcatggataaacctttatttctataaccattaattacggaacagaatgtagagctgttgtaacattatttccaatgaatatattaagggccgcgccctaagtgaatctgaattattttggtaattagctgtctgtcacgtttcctcattgctatgaattactttaggtgacacatctgtgcatatatgagtgacatctcaaattacagcattcttcaccccgggagcactgaaaaacaagtacatacgaatattggcggtgaaagtttctcaaattgataaggaggtaaacttacgggttcaccaacattgtggcattttgctgccgaatcaatcttaaaaaaaacattcgatggttctgactcactaggtagaccaataactttcacctcatcattgtttgaatgtgcccgttgttttttgatactctgaaattcactggcactcaaatatgtttctttaagacggtgcgcaggatagccacaacgtgagccgtaggttaagcattttccgctgcagcaatctgtatgcatgttgcactggaatttagaaaatatccgtaatatgtatctaggatctaggattcatacacaaaacttacatttccaaagactggactacatttttgcccataagcatccacattgctcagcataaagaacaaaagacacaatccaatgatcgtagaaatcgacgacat

SEQ ID NO: 19 - DNA, 552 bp, Drosophila melanogaster
atgtcgtcgatttctacgatcattggattgtgtcttttgttctttatgctgagcaatgtggatgcttatgggcaaaaatgtagtccagtctttggaaattgcaacatgcatacagattgctgcagcggaaaatgcttaacctacggctcacgttgtggctatcctgcgcaccgtcttaaagaaacatatttgagtgccagtgaatttcagagtatcaaaaaacaacgggcacattcaaacaatgatgaggtgaaagttattggtctacctagtgagtcagaaccatcgaatgttttttttaagattgattcggcagcaaaatgccacaatgttggtgaacccgtcggatgggcaataattgtggagtttaaaccactgcagcagcagcttaatcctctcaaccaggatgaggttaccatgttcaccaacatgtttctcagcctgtttcgcggagtagccgactatcgaaacatggtggctgccgattcagtgcagagtgtttggcagagtccgccagttgttgactttagtcaccgactctttgccagctag

SEQ ID NO: 20 - protein, 27 aa, Anopheles gambiae
CKAIGDSCTRHENCCSSNCHSYRGKCV

SEQ ID NO: 21 - protein, 28 aa, Coremiocnemis validus
CSRAGENCYKSGRCCDGLYCKAYVVTCY

SEQ ID NO: 22 - protein, 27 aa, Drosophila melanogaster
CSPVFGNCNMHTDCCSGKCLTYGSRCG

SEQ ID NO: 23 - protein, 27 aa, Drosophila melanogaster
CQPSGGYCKSHADCCSTMCLTQLGQCS

SEQ ID NO: 24 - protein, 27 aa, Anopheles gambiae
CAKNNEYCLTHRDCCSGSCLSFSYKCV

SEQ ID NO: 25 - protein, 27 aa, Anopheles gambiae
CALNGEYCLTHMECCSGNCLTFSYKCV

SEQ ID NO: 26 - protein, 27 aa, Anopheles gambiae
CAKIGEYCLTSSECCSKSCLSFAYKCV

SEQ ID NO: 27 - protein, 25 aa, Anopheles gambiae
CTSNGLYCVHNKDCCSGACYKSVCS

SEQ ID NO: 28 - protein, 30 aa, Agriosphodrus dohrni
CLPRGSKCLGENKQCCKGTTCMFYANRCVG

SEQ ID NO: 29 - protein, 30 aa, Isyndus obscurus
CLPRGSKCLGENKQCCEKTTCMFYANRCVG

SEQ ID NO: 30 - protein, 30 aa, Peirates turpis
CIAPGAPCFGTDKPCCNPRAWCSSYANKCL

SEQ ID NO: 31 - protein, 30 aa, Drosophila pseudoobscura
MPCDSCGKECANACGTKHFRTCCFNYLRKR

SEQ ID NO: 32 - protein, 30 aa, Anopheles gambiae
LSCDSCGRECASACGTRHFRTCCFNYLRKR

SEQ ID NO: 33 - protein, 30 aa, Tribolium castaneum
QSCTSCGSECQSACGTRHFRTCCFNYIKKR

SEQ ID NO: 34 - protein, 30 aa, Bombyx mori
  misc_feature (18)..(18): Xaa can be any naturally occurring amino acid
LSCDSCGNECTSACGTSXFRSCCFNYLRRK

SEQ ID NO: 35 - protein, 22 aa, Apis mellifera
DQCGRKCANICGTQQFPACCFN

SEQ ID NO: 36 - DNA, 252 bp, Drosophila pseudoobscura
agctcaaatgccatgccctgtgactcctgtggcaaagagtgtgccaacgcctgcggcactaagcattttcgaacctgctgctttaactatctacgcaaacgcaatgatccggatgagctgcgtcgcagctccgatcggagactgattgacttcatattgctgcagggcagggccctgtacacccaggagctgcgcgagagacaccacaatggcaccctgatagacggcaccctcggcctgcagacctactat

SEQ ID NO: 37 - DNA, 189 bp, Anopheles gambiae
aacatgttttcttttcattggattatgtggtattatttgataaatttggtaagttgtgtattgtgggtggcctgtagtttcccagtagctctctcgtgcgattcatgtggtcgggaatgtgcatctgcatgcggtacaagacactttcgtacatgctgcttcaattaccttcggaaacgtagctcacca

SEQ ID NO: 38 - DNA, 1001 bp, Apis mellifera
ttaaaatataaaaattagaaaataaacatatttcttacatctgtaatatatatattatggaaaaaattatcctatgaccaatgtggcagaaaatgtgccaatatatgtggaactcaacaatttcctgcttgttgctttaataatataaaaaaaaaaacaatttgatttagaattaaaagtcaggatgaaatcgcatcgatgtacaattgtatctgaatattaatatacaatacttaatatactaattatttaatatattttatcatctatattaatattattgtcattaatattattttgtcattttatatgtgttttaaaagtaaatatatgtatatattatatgctatatataattaaggattatttttaattgtaattataaatattttaattttgcaatatatacgaaagacatttcgtcaataaacggaagtataaagattaattttatttttttaagtagaactataatttttttaatataaataaatacaacaaattattctttataaaaaaaaaactattaaatgatagaaccaaaaactattaatttaactgttattattaaatatatagaattaaaaatatatagaaaaatatatttaaaaatatattaaatatcttataaactaataataattatttacaaaaataaagttgacttcagaagttttttacgcattttcattcgaaaaaaatcaatgacatcagaatataatcctgaaattattcaacttttatctaaaatatttttttctataaacgataattctgacgtaaatgaagataataatattagatgatcatattaatattatctttcttagatattatattaataaaactatgcaaaactatgcaaaagatataaaataagaataaaaatttaaaaatatataaaaaatatataataatacataatacaaatatataataaaacatataaaaaaatatatgaatatatattttatatatatcttctgcaatttatcaagtta

SEQ ID NO: 39 - protein, 111 aa, Mus musculus
MFVLVMICLFCQYWGVLNELEEEDRGLLCYKCKKYHLGLCYGIMTSCVPNHRQTCAAENFYILTKKGQSMYHYSRLSCMTNCEDINFLSFERRTELICCKHSNYCNLPMGL

SEQ ID NO: 40 - protein, 106 aa, Mus musculus
MKNFLRLCLFLLCFETGFPLQCVQCQSYKNGECATKKETCTTKPGETCMIRRTWYANEIHNLQDAETKCTNSCKFEEKTSGYLTTHTYCCSHGDFCNDINLPIVMT

SEQ ID NO: 41 - protein, 117 aa, Mus musculus
MGKLLLLHFLLMQASFALVFIQVQATVCMVCKSFKSGHCLVGKNNCTTRYKPGCRTRNYFLFSHTGKWVHNHTELDCDKACMAENMYLGALKISTFCCKGEDFCNKYHGQVVNKNIY

SEQ ID NO: 42 - protein, 168 aa, Mus musculus
MHMLIYYQFLHLFQFPWCACWIPLHTCSAEDEASLCCFCCCCCCFVLFLFVLLIILFIYISNYFSLHRGSNSYNLYASFFPLSWMLTPSYPTSDTKHSPFIFISCLSSFICENHHQSCLSCIYLSLTITKLLWLTSYQASNLNIISMSQILQKSYIPNRQCSLLFLVC

SEQ ID NO: 43 - protein, 137 aa, Mus musculus
MFQKLLLSVFIILLMDVGERVLTFNLLRHCNLCSHYDGFKCRNGMKSCWKFDLWTQNRTCTTENYYYYDRFTGLYLFRYAKLNCKPCAPGMYQMFHDLLRETFCCIDRNYCNDGTANLDTSSILIEDMNQKKELNDD

SEQ ID NO: 44 - protein, 136 aa, Mus musculus
MFQKLLLSVFIILLMDVGERVLTFNLLRHCNLCSHHDGLKCRNGMKSCWKFDLWTQNRTCTTENYYYYDRFTGLYLFRYAKLNCKPCAPGMYQMFHDLLRETFCCIDRNYCNDGTANLDTSSILIEDMNQKKELND

SEQ ID NO: 45 - protein, 99 aa, Mus musculus
IRMYILLHLLGLSFLVGFLKALTCITCDRINSQGICESGEGCCQAKPGEKCASLITLKDGKIQFGNQRCANICFTGTVQTGDQTVKMKCCKKRSFCNEL

SEQ ID NO: 46 - protein, 101 aa, Mus musculus
MNPVTKISTLLIVTLPFICFAEALKCFQCTLFNSKGKCLFQEPPCETQNNEVCVLWAKFEGGRFMYGFQECSHTCVNQTLNLRNKRIEMKCCNDKSFCNKF

SEQ ID NO: 47 - DNA, 2216 bp, Mus musculus
ggagaaaagagccagtaccttcctcagaaagctgctgaacacaagggttgcaggatgtttgtgctggtgatgatctgtctgttctgccaatattggggtgtccttaatgagcttgaagaagaagatcgtggactactgtgttataaatgtaagaaatatcatcttgggttatgctatggaatcatgacatcctgcgtaccaaatcatagacagacctgcgctgctgagaacttttacatactcacaaaaaaagggcagagtatgtatcattattcaagactgtcatgtatgaccaactgtgaggacatcaacttcctgagttttgaaaggaggacagagctcatttgttgtaagcacagtaactattgcaacctcccaatgggactctagttctgaatttattatggatttggtatcattcttcaacttactaccaactctctttcctccaaagtttgtatttactctcccctcctaacttactaaaaattggaaaatcatttgtcagtgaaaagagaagcagtcatatgagaaactggctgggagctcagcctcattgctaagcaaatctttgcaaattctttttgtcatatctagcagggcattttgatctgtggacaatactgcccatcatgagtaggtccagaattgatcatctcatataccaagccacaagattttggctcaaaatgacatcccaactttgtacagggaaatctgtaaatatactgtgttggtctgcaccaagtcttctgagttaagattttgtttggactagctctgagaattcttggcacagtacttatggattcaggaggtaagagagtgttaatcccagcttcccaatttgctaatgatctgagactttttttttggcaaatctcatgggagaaatatgagaggtgagaaaaatctggataagcacagatattaaacaacaaaattagaaatgtatggaaattttcattgatgcagagacagtgtgatgcatctggatctgattgtattgaggctgtggttcaatcaggtgattggattaggggttcaggccttgttcaaaagtgattactcaatattctgatgaacgtgaagaaaaaaaactggatttctatagtagttaaaggtaacagaatatgtaaagatagcatgagctatcatgtactagtttcctttatgttgctgtgataaaacactttgaccaataccaactgagggtaggaaagggtttatctagtttatacttctagataacagttcatcatggagtgcggtcaggacaggaactcaaggtaggaacctggaggcaggactcatagagggacaatgcttgtcacaggcttatgcttagcttgctttctcatacaatacaggaccaactgtcttgaaatggtactatcaacagtggtctggaccctcttatgtcaataagaataatcaagattgttctccacagacacgtccacgaggcaatttgatctagacaacttcttttttgtttttatttatttatatttatttggttttattaatttactttatatcctgactatagttcctggccttccagttccccctccccccattcactcctcctccatttatcttcagaaaatatgtatctaccacttatgaagttgtagtaagactttgcacatcctctcttattgaggccaagacaagacaggcagtaggagaaaagggtcccaaaggcaggtgacagaatcagagatatctctaggcaactttttaattgaggtcatatccctgtaggttgtgtcaagtagacaaggttaacatggtgacatgatatggtgtttgggtaatactgtgtgccacagtagttaaagttatgaagagcagaaagtgtgacagaatagaaaggtaacattagtaagaggggaaacatacagttattacatgatcaagtaccaaggtataaacaaaaccagaacagcacagacactggagcatcacatattggggcaagataggcagacggtaaccacgtttctctggtttggaagcctattgcaattcctgtttctggattcctatgatcattctcaactatattaagccacataagattctccaatgaacaaactacttctctatttgaagagttaaatgaacttccatctttgtcttgtatctgagaatgtcatttttacttctttttgaggaattcccaatttggactcttagaaccgggatgg

SEQ ID NO: 48 - DNA, 1334 bp, Mus musculus
ataaattcactagaggctggtcttttctcagggtcatctctgatcatcagctgtccggtgctaatagagccggctagaagaatctgctcaggagaaatgaaaaacttcctgaggctgtgtctctttcttctctgctttgaaacaggattccctctacagtgtgtgcagtgtcagtcttacaaaaatggggagtgtgccacaaaaaaggagacatgcactacaaaacctggtgaaacgtgtatgatccgcagaacctggtatgcaaatgaaattcataatctgcaagatgctgagactaagtgcacgaattcttgtaaatttgaagaaaaaacttctggatatctaacaacacatacatactgctgtagtcatggagatttctgtaatgacattaatttacccatcgtgatgacttagcataatctagttccagttgacctcatcagccctttccttttcattctccattttctttcattgacttttattttcaatctggactatagatttttctgataatcaatattgtgcaagtcatagaacctggggacatacagaaatttcttgttcttttgaaagtatgtttgatcatatagtctaattcttgtgcttggtatccctagacttattaccatgaatatagctaaatcttggtttccattaatgtatttatcacttcagtgtgggtggctaaaataaataaatggattaaaatatgtgaactgtcaggaagagtgtgaacattcccagtatcctttgcataacatgtactttatgaacttagaattgttctcattgtgacttacacttccaatatacagtttaaaggaggaaagattgctgtggattctgcttttaggctttcactgtcttgttccatggctatggacctgtgataaggctgaatttcatgacagcaaaaggtcatggctcaccaaaacttctcacccactggcatctaaaatgtagtagtacaaagggaggaccagggcaaaatatgaaatacactcagtagcttatgcttgctaagtatgctgaaaagaaaggcatctgaataaacgtgtttccatgaatgacaaaaatcactgctacaaagttatagtctaactttcacccaattaaaaaaatctccacctactgcttctgatatattggtgccagaaaatgaatccactaagtgtttcttattctttgaaagatgttctttctgagcatgtataacatgaatgaaatgttgtttacattcacctgtcttaagaaagtaatactacaccaagttgtctttgtgtcattaaaatggaattgcttctgagccttcattcccttctccatct

SEQ ID NO: 49 - DNA, 443 bp, Mus musculus
agaatctgtaacctgctctcatctacactgaccgtcctgagcacttgctaccagctgctctcctgtgtcctctgatatcccagactgagatgggcaagctcctgctcctacactttctgttgatgcaggcatcttttgctcttgtgttcatccaagtccaagctacagtgtgcatggtctgcaagtcttttaagagtggacattgtttggtaggcaagaacaactgcactacaagatataagcctggatgcagaaccaggaattacttcctattctcacacacaggtaagtgggtccacaatcacaccgaattggactgtgataaggcatgtatggctgaaaacatgtatcttggagccttgaagatatctaccttttgttgcaaaggtgaagatttctgtaataaatatcatggccaagtagtgaataagaatatttactaa

SEQ ID NO: 50 - DNA, 507 bp, Mus musculus
atgcatatgctaatttattaccagttccttcatctgtttcagtttccctggtgtgcctgctggatccccctccatacatgctctgctgaagatgaagcaagtctttgttgtttttgttgttgttgttgttgttttgttttgtttctttttgttttattaatcattttattcatttacatttcaaattatttctctcttcacagaggaagcaactcatataacctctatgcatccttctttcctctgtcttggatgctgactccttcctacccaacatcagataccaaacattctccattcatcttcatttcctgcctttcttcatttatatgtgaaaatcatcaccaatcttgcttgtcttgcatttatctctccctcacaatcactaagttgctctggctgacgagctaccaggcttctaatttaaacattatttcaatgagccaaatattacaaaaatcttatattcctaatagacagtgttctctgctttttttggtgtgttaa

SEQ ID NO: 51 - DNA, 908 bp, Mus musculus
aggtcagaaggaggcccaattatgttccagaagcttctgctgagtgttttcataattctccttatggatgagaaagagtgctgacatttaactgtacagtatatttggcttgcatttattggaaaaatcatactaccatacgaggagaagtttttgaaccatttacacactgttcctctccgtacctcattcactacttctgagtcttctttctatagatttgatttgtgaatgagtagggaactcttggagggaatcattactgccattaataatatcagggggtgggggaagagtgccacatttcttctcattgggtttgtataattttgactttctaaagtagttattttccacagatctaacattaaaaatcttcctttcctttagtgcttagacattgtaatctgtgttcgcattatgatgggtttaaatgccgcaatggcatgaaatcatgctggaagtttgacttatggacacaaaacaggacttgtaccacagaaaactattattattatgatcgtttcacagggttatacctttttcgttatgccaaacttaattgtaaaccctgtgcacctggaatgtatcaaatgttccacgacctgctgagagaaacattttgctgtattgacaggaactactgtaatgatggcactgctaacttggatacctcatcaatacttatagaggatatgaatcaaaagaaagagttgaacgatgattgaaataatgaggatttaaatacctcatgtgcctatattcttgacaattataaaacccaggccccatactcctctctatgtcagtaaatgttcccatgcaaacccagtcttttttatttccacatttcaaataataagaaagagaaaactcacaagtaaaaacaaaacaaaacaaaacaaatc

SEQ ID NO: 52 - DNA, 1155 bp, Mus musculus
gtcagaaggaggcccaattatgttccagaagcttctgctgagtgttttcataattctccttatggatgtaggtaaggcctggaaagaaaagagaatgttatgctataggaggaaaaggtttttatacttcactgtggggctactatgaaatgacttaaggggaattttccttctcttcacaggagaaagagtgctgacatttaactgtacagtatatttggcttgcatttattggaaaaatcatactaccatacgaggagaagtttttgaaccatttacacactgttcctctccgtacctcattcactacttctgagtcttctttctatagatttgatttgtgaatgagtagggaactcttggagggaatcattactgccattaataatatcagggggtgggggaagagtgccacatttcttctcattgggtttgtataattttgactttctaaagtagttattttccacagatctaacattaaaaatcttcctttcctttagtgcttagacattgtaatctgtgttcgcattatgatgggtttaaatgccgcaatggcatgaaatcatgctggaagtttgacttatggacacaaaacaggacttgtaccacagaaaactattattattatgatcgtttcacaggtaagcaagcctttgaaaagcacatgcaaaaatgtctccagttctacccacacaatttggacttaagatgcagagggcagttcagatctcataggttcttaagacagagagcaaggcataactctggaggaagacaggcattttggctgaatataactagagatatatagatgagatgcagccacagccctgggctcatattaattgcaaaaaaaatattatgaggttctagaaggaggcatgttgatgacattcgtttttcttctttttagggttatacctttttcgttatgccaaacttaattgtaaaccctgtgcacctggaatgtatcaaatgttccacgacctgctgagagaaacattttgctgtattgacaggaactactgtaatgatggcactgctaacttggatacctcatcaatacttatagaggatatgaatcaaaagaaagagttgaacgatgattgaaataatgaggatttaaatacctcatgtgcctatattcttgac

SEQ ID NO: 53 - DNA, 594 bp, Mus musculus
atgttccagaagcttctgctgagtgttttcataattctccttatggatgtaggagaaagagtgctgacatttaacttgcttagacattgtaatctgtgttcgcattatgatgggtttaaatgccgcaatggcatgaaatcatgctggaagtttgacttatggacacaaaacaggacttgtaccacagaaaactattattattatgatcgtttcacagggttatacctttttcgttatgccaaacttaattgtaaaccctgtgcacctggaatgtatcaaatgttccacgacctgctgagagaaacattttgctgtattgacaggaactactgtaatgatggcactgctaacttggatacctcatcaatacttatagaggatatgaatcaaaagaaagagttgaacgatgattgaaataatgaggatttaaatacctcatgtgcctatattcttgacaattataaaacccaggccccatactcctctctatgtcagtaaatgttcccatgcaaacccagtcttttttatttccacatttcaaataataagaaagagaaaactcacaagtaaaaacaaaacaaaacaaaacaaatc

SEQ ID NO: 54 - DNA, 408 bp, Mus musculus
atgttccagaagcttctgctgagtgttttcataattctccttatggatgtaggagaaagagtgctgacatttaacttgcttagacattgtaatctctgttcgcatcatgatgggttaaaatgccgcaatggcatgaaatcatgctggaagtttgacttatggacacaaaacaggacttgtaccacagaaaactattattattatgatcgtttcacagggttatacctttttcgttatgccaagcttaattgtaaaccctgtgcacctggaatgtatcaaatgttccacgacctgctgagagaaacgttttgctgtattgacaggaactactgtaatgatggcactgctaacttggatacctcatcaatacttatagaggatatgaatcaaaagaaagagttgaatgat

SEQ ID NO: 55 - DNA, 300 bp, Mus musculus
attagaatgtacatcctgctgcacctgctaggactctcttttctggtgggattcctgaaagctttgacatgtatcacgtgtgataggatcaattctcaggggatttgtgagagtggagaaggttgctgtcaggctaaacctggagagaaatgtgcctcgctcataacccttaaagatggcaaaattcagtttggaaaccaaagatgtgctaacatttgcttcactgggactgtgcagactggagatcaaacagtaaaaatgaagtgctgcaagaaaaggtctttctgcaatgaactataa

SEQ ID NO: 56 - DNA, 2343 bp, Mus musculus
ggtgcctatgttggagattccttcctggtctttagctctataaagagagcgaatggtcatactttacctcaagttgccttctaacatccaacatgaatccggtgacaaaaatcagtacactgcttatcgtgactttaccctttatctgctttgcggaggctctgaaatgcttccagtgcactttgttcaactctaaggggaaatgtttgttccaagaacccccctgtgagacccaaaataatgaagtatgtgtcttgtgggcaaagtttgaaggtggcaggttcatgtatgggttccaggaatgttctcacacttgtgttaatcaaacactgaacttgagaaataaaagaattgaaatgaaatgttgcaatgacaaatctttctgcaacaagttttagaagcataaaccatcttgacatgttccaaggacagttctgagcccttcatcctcctcctgtgccctcacaacactgcaccatctattccagcactgcccatgctctgatacctgcttgtactctgcaccaggtgtggctatttggacagttcagctggggatagagaaatgccctgtggtccccacaaccctcagtgcttctccctcttttgtagatcctgatttccttccttctcaccttctagtcttccaggactgacagattgcacactcttctgctgctcatactgacttgtttttgtcaaatgcatctgataactaatactgttgttaggtgctagttctccaacagtgactatggcagagatgacacctgcccttgatgaccagttcagtgatgcatttttctattcggttggccatcaaacaatagctctagcatcttactgcagttttattcatagaatttcatgtgtgtgtcagggagttttgcactgctgtggcctgagtagaacaacataagggacacctaggtgaggtggcatatgccttaaaccccagcacttgagaggcaaaggcaggaggatttctatgagtctgaggctagattgttctacttagcaatagtgcctcaacacacacacacacacacacacacacacacacacacacacacacacacacacactcacacacacacacacactcacacatgcacgcacatgcacacatgtgcacacattttccccttcagagcacctgtgtcctactagctccatctctggagagtacttgagttcacataatttcatttaatctctttttatgttgtgaatattttcctccagtcaataactttttagatgctcaacattgcctttccaagagcaacagacattgatttttaataatattgtctgtagtttcattaagttgcttcgctaaaacactgtgatggaaaacaatttagaggtgagcttgtttatgtaaccttagaggctacagtgaggctctaatgtgtcaccaccagcttctcctatggactggatgtgtgagttaccagagatccatatgttcaagtttagtccctgagatggtgaaatttggaagtgaggcttttgggaggagattaagttatgagggtagacccataacaaatgagactcatgcccttatctaagagacaaatacaggaattcctagtatttatgtatttatgaatatacatatacataaatatccatatatttatgtaacaacagtgaaaaagaggccatgaatttgaaaagaagcaagggagatataagggaaagtttggagggagaaaagaaagaggaatttatagttataagaaaaggagtataattatgctataatctcaaaaaatgaaaaatgatttaaaattttcttgttcagaagctggatagtctattcattttctttcttttctcttcagcgtcctaaatgaactgaggcaacgacttatctaagatatcaaaattatttgctaacgtcaaaattcttagagaatacaaatactcctttatttctgtaattttaaactttggagttttatatttgttttgtcatgcattttacttaatgtgtatactatttatggtggatcaaagatattttgtttctttattttttgcccatgaatgtccaattatcttttgataatttgtttgaaactcctttcttctcataattataaaggccaagtgtccacatccatgtaagtttatagcaaaattctatatttttctttctttcattatatcccactattttccttgataactgtagagtttttggacctttaaaatcagacaatgtgtcttctaattttgctccttttttaaaagttaatttgagtattttaaggtctttgaattgctatagatattttcagatcatcgtattgatttctaataaaatgattg

SEQ ID NO: 57 - protein, 126 aa, Homo sapiens
MDKSLLLELPILLCCFRALSGSLSMRNDAVNEIVAVKNNFPVIEIVQCRMCHLQFPGEKCSRGRGICTATTEEACMVGRMFKRDGNPWLTFMGCLKNCADVKGIRWSVYLVNFRCCRSHDLCNEDL

SEQ ID NO: 58 - protein, 113 aa, Homo sapiens
MLVLFLLGTVFLLCPYWGELHDPIKATEIMCYECKKYHLGLCYGVMTSCSLKHKQSCAVENFYILTRKGQSMYHYSKLSCMTSCEDINFLGFTKRVELICCDHSNYCNLPEGV

SEQ ID NO: 59 - protein, 95 aa, Homo sapiens
MNTLLLVSLSFLYLKEVMGLKCNTCIYTEGWKCMAGRGTCIAKENELCSTTAYFRGDKHMYSTHMCKYKCREEESSKRGLLRVTLCCDRNFCNVF

SEQ ID NO: 60 - DNA, 1576 bp, Homo sapiens
cctctttccaaaatggacaagtccctcttgctggaactccccatcctgctctgctgctttagggcattatctggatcactttcaatgagaaatgatgcagtcaatgaaatagttgctgtgaaaaacaattttcctgtgatagaaattgttcagtgtaggatgtgccacctccagttcccaggagaaaagtgctccagaggaagaggaatatgcacagcaacaacagaagaggcctgcatggttggaaggatgttcaaaagggatggtaatccctggttaaccttcatgggctgcctaaagaactgtgctgatgtgaaaggcataaggtggagtgtctatttggtgaacttcaggtgctgcaggagccatgacctgtgcaatgaagacctttagaagttaatggttcttctgtgactccaatttctgggtgaggttgttgcctcagcctcttcacaatgactttctaaaaaaaatcacacacacacacacacacactacagaagaggattgcaaacacatggctccatcttctgcacacgaaaggaaagtccctctccttttctacagtctctgtcacgccccttaaaataagtaaataaataaccttgagagaaagaacaagatcaatatatcctgcaggttgctacaaacccttgtgctttcactgtatagccagttcattcagaaaaggaggaaagggtagtttaatttcaaaaaagaatcccttcctctttcctctgctgctttccttccttctgtggcagggtattttaatatatttttcaaatttttttcctttctgtgttatccttcttatcccactccaaagaaagcacataactgtggcctgaagggatggggagtagcaacataaaaagaagtggctcaagtcttcttggagtttgttcatgaatgctgatcccagggtgaggagaagattgggacatagaaaggaaactgcatcagaaacatgaacagagaaagattgtctgccttctagaatcagatctgtttggggctgggggttggagaataaaagcaggagaagtctatgggattctagaaatagtacctgcatccagcttccctgccaaactcacaaggagacatcaacctctagacagggaacagcttcaggatacttccaggagacagagccaccagcagcaaaacaaatattcccatgcctggagcatggcatagaggaagctgagaaatgtggggtctgaggaagccatttgagtctggccactagacatctcatcagccacttgtgtgaagagatgccccatgaccccagatgcctctcccacccttacctccatctcacacacttgagcttgccactctgtataattctaacatcctggagaaaaatggcagtttgaccgaacctgttcacaagggtagaggctgatttctaacgaaacttgtagaatgaagcctggaaagagtgatgaattatattatattatataaaaataataataaaaatataaagaaagctaaaaaaaaaaaaaaaaaaaaaaaaaaaaaaaaaaaaaaaaaaaaaaaaaaaaaaaaaaaaaaaaaaaaa

SEQ ID NO: 61 - DNA, 1690 bp, Homo sapiens
caacttgctccttccacaggaagctgcacctgacagaagctccaggatgcttgttctctttctcctgggcacagtctttctgctctgcccatattggggtgaacttcatgaccctataaaagcgactgaaataatgtgttatgaatgtaaaaaatatcatcttgggttatgctatggtgtcatgacatcctgctccctgaagcataaacagtcctgtgcagttgagaacttttacatccttacaaggaaagggcagagcatgtatcattattcaaaactgtcgtgtatgaccagctgtgaggacatcaacttcctggggttcacgaagagggtagagctcatctgttgtgatcatagtaactactgcaacctccctgagggagtttagttctacgtctctcctggattttgggttctttttcaaccactacgctctttttctcttccctgaacctgaattttgctctcctcttctatgcattggtagagagtgagaaagccagctcatagtgaaaagacaagcaggcataaggagacgcagtgcggacagcggagcctattgatgatggagcactagactcactctttgcacatccctgtcgctaacaggtgggaggggttttgctctccttacgtgatactgccatgaataagctcagacttggtcatttattatctcctgtatgaaaatgtgaacacttgggccataataatctccaatttgtactgagaattctgtgactatcctctatcctcatctacacacacactcctctccgttggaattctctttggattagccctgacactttctggcactgtccttttctgcccgtgggttctggagagggctaaccccagcttcccagtgtgttggcagcatgggccaagtctttctctgatagagctcttgggggaacctcagggcagaaaaaaaaaactgaacagacatagggatgagcatcaaagaaaacttcagggggcatttcaactggtaaaaactaagatctgagaaataacttgctgtgggtggaattggcttgaattatcattgccctggctggccaggcagccttgggcacatggttgaactaggtgattggattgggaacagagtgtttttttaaggatggctagtcaggttcttctcatgggactgagcaaacaaaatgctgataatgtggcaggtggtgtagtagttgaagataataggatgtaaatagataccatgaggtagtgttcaagtaacagagtgccctgtgtaagaggaagttatgagtttgcagaaagtgtgagaaaggtagcatatatgggtaacagtggtgggaagagaagaaggaagacacagaaacagataggaagagaaactgagtagcattgatgttggagcacacctgtggccaaggtggcacattttccttggatacacactaaccctggcgtttctgtccttataaggctgactgcaccctagatgacgggttgatgggtgcagcaaaccaccatggcacatgtatacctatgtaacaaacctgcatgctgcactttctttcatgtatcccagaacttaaagtaaaataaaaaaataaataaataaataaaggccagaaaaattggccaaaaaatctgaatagaaaaaaaaaaaaaaaaaaaaaaaaaaaaaaaaaaaaaaaaaaaaaaaaaaaaaaaa

SEQ ID NO: 62 - DNA, 1983 bp, Homo sapiens
aatacctcactcagcacaccgtctgtcacccaaacaagcatccaatgaggaaaatgaacacactgctccttgtgagcttatcttttctctacctcaaagaggttatgggtctgaagtgtaatacctgcatatacacagaaggatggaagtgtatggcaggccgaggcacttgcattgcaaaagaaaatgagttatgttcaacaacagcctatttcaggggagacaaacatatgtactcaacacatatgtgtaagtataagtgccgggaagaggagtcctccaaaagaggcctgttgagagtgacactgtgctgtgacagaaacttctgtaatgtcttctaatggagcttaggaacttgcagaggatcatctgatcaagatccagaatcaagaccaaccaacatgaactgttttatttcccacaccaaattccacactggcctaagatcccagagagagctgcaggggctgtcctcattgcaatgaaggggctccccacaccccacctccaccactagattcctaaaatcatgagcattgaaacaaaatcctccatgagctatgcttattcttttgtcttctactcctgatttctgatttctatcccttgtaggctaaataatggacccccaaatatttctacatcctaatctctggaacctgtgaatgttaccttacatggcaagagggactttgaaaatgtgattaaggattttgagaaggggaggttatcttgcattatctgggggggccctaagtggaatcaaaatgcaaaatgcaataagatgcaagtaaagggagatatgacaaagaagaggaaaagatgatgtgataatggaagcagagattggagccatgtgctttgaagatggaggaataggccataagctaaaactaggaggccactagaagctgaaaaagtcaaggaaataggttctcccctcagagcctccagaaaccagccctgctgacaccttgatttaagccctgtaagactcattttggatgtctggcctccagaactctaagcgggtgtggttttcagccacaaagtgtgtattgttttaagccactaagtttgcaataatttattatagcagcaatgggaaactaatacaatccaaataaacttctaggaattcaaatcattggtaagcctgagtacccaggggccagtctaggtgacaacagtatccaccgctcagggcttacagtgacctgcaggaagagaggaataacagagcacatgctatgaataaatgtggagatcaatttgtggattttaaacttcatgacatgctgcagataatcctttaggtgtatctgtggtaaatggtgccttgctatgttctgaagcaatcaaacatgatgtcctaatagctcaatttatcagttcctcattagcttgtgtactcactgaaattcttttagcagttaatgtccttgtattagtctgttttcatgctgctgataaggacatacccacgactgggcaatttacgaaagaaaggggtttatcggacttacagttccacgtggctggggaagcctcacaatcatggcagaaggtaaaaggcatgtctcacatagtgacagacaagagaagagagattgtgcaggaaaactctcccttataataaccatcagatcttgtgagacttactcactatcacgagaatagcacaggaaaaacctgcccccatgattcaattacctcccaccggtccctcccacaacacgtgggaattcaagacgaaatttgggtggggacacagccaaaccatatcagtccccttcttaaaactctcctctctagctcctatgactgtacatttgtagttctcccacctctctgaagaatcctctgtgagggtctctggggactttgttttgatttgttattgtttttatattcagtctttaacctatgtgaagccacctttgtaatactgccttaagtaaagatccagtattatttttctcc

SEQ ID NO: 63 - protein, 27 aa, Apis mellifera
  misc_feature: OCLP1 active sequence, missing the last amino acid
CGRHGDSCVSSSDCCPGTWCHTYANRC

SEQ ID NO: 64 - protein, 26 aa, Apis mellifera
  misc_feature: OCLP1 active sequence, missing the last two amino acid
CGRHGDSCVSSSDCCPGTWCHTYANR

SEQ ID NO: 65 - protein, 25 aa, Apis mellifera
  misc_feature: OCLP1 active sequence, missing the last three amino acid
CGRHGDSCVSSSDCCPGTWCHTYAN

SEQ ID NO: 66 - DNA, 21 bp, Artificial sequence
  Single strand DNA oligonucleotide
tcatgtccaagtttattcttc

SEQ ID NO: 67 - DNA, 24 bp, Artificial sequence
  Single strand DNA oligonucleotide
aggagctcttaacacctgttcgca

SEQ ID NO: 68 - DNA, 21 bp, Artificial sequence
  Single strand DNA oligonucleotide
cttaatctttcccctttctgc

SEQ ID NO: 69 - DNA, 24 bp, Artificial sequence
  Single strand DNA oligonucleotide
aggagctcttaacacctgttcgca